SEQUENCE LISTING (11 sequences)

SEQ ID NO: 1 - protein, 10 aa, Artificial Sequence
  synthetic construct
GYKFTRYVMH

SEQ ID NO: 2 - protein, 17 aa, Artificial Sequence
  synthetic construct
YINPYNDGTNYNEKFKG

SEQ ID NO: 3 - protein, 8 aa, Artificial Sequence
  synthetic construct
ARNWDTGL

SEQ ID NO: 4 - protein, 11 aa, Artificial Sequence
  synthetic construct
KASDHILKFLT

SEQ ID NO: 5 - protein, 7 aa, Artificial Sequence
  synthetic construct
GATSLET

SEQ ID NO: 6 - protein, 9 aa, Artificial Sequence
  synthetic construct
QMYWSTPFT

SEQ ID NO: 7 - protein, 115 aa, Artificial Sequence
  synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSS

SEQ ID NO: 8 - protein, 107 aa, Artificial Sequence
  synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIK

SEQ ID NO: 9 - protein, 441 aa, Artificial Sequence
  synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, Artificial Sequence
  synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 345 bp, Artificial Sequence
  synthetic construct
caggtgcagctggtgcagtctggggctgaggtgaagaagcctgggtcctcggtgaaggtctcctgcaaggcttctggatataaattcactcgttatgttatgcactgggtgcgacaggcccctggacaagggcttgagtggatgggatatattaatccttacaatgatggtactaactacaatgagaagttcaaaggcagagtcacgattaccgcggacaaatccacgagcacagcctacatggagctgagcagcctgagatctgaggacacggccgtgtattactgtgcgagaaactgggacacaggcctctggggccaaggcaccactgtcacagtctcctca

(19) United States
(12) Patent Application Publication
(10) Pub. No.: US 2023/0322913 A1
(43) Pub. Date: Oct. 12, 2023

(54) Therapeutic Antibody Formulations

(71) Applicant: Eli Lilly and Company, Indianapolis, IN (US)

(72) Inventors: Aaron Paul Markham, Indianapolis, IN (US); Galen Huaiqiu Shi, Zionsville, IN (US); Justin Cody Thomas, Fishers, IN (US)

(21) Appl. No.: 18/044,504

(22) PCT Filed: Sep. 10, 2021
(86) PCT No.: PCT/US2021/049773

§ 371 Date: Mar. 8, 2023

Related U.S. Application Data
(60) Provisional application No. 63/076,600, filed on Sep. 10, 2020.

(51) Int. Cl. C07K 16/24; A61P 37/02

(52) U.S. Cl. CPC C07K 16/244; A61P 37/02; C07K 2317/56

(57) ABSTRACT

Stable pharmaceutical formulations for therapeutic anti-IL-23p19 antibodies and methods of using such stable pharmaceutical formulations.

The present invention is in the field of medicine. More particularly, the present invention relates to aqueous pharmaceutical formulations comprising therapeutic antibodies that are suitable for subcutaneous (“SC”), intramuscular (“IM”), and/or intraperitoneal (“IP”) administration. Still more particularly, the present invention relates to pharmaceutical formulations of an anti-IL-23p19 antibody. These anti-IL-23p19 antibody pharmaceutical formulations are expected to be useful in treating at least psoriasis (Ps), psoriatic arthritis (PsA), ulcerative colitis (UC), Crohn's Disease (CD) and/or ankylosing spondylitis.

Pharmaceutical formulations of anti-IL-23p19 antibodies are needed for the treatment of patients with Ps, PsA UC, CD and/or ankylosing spondylitis. Administration of such therapeutic antibodies via SC, IP and/or IM administration is both common and advantageous. Such routes of administration allow the therapeutic antibody to be delivered in a short period of time and allow patients to self-administer therapeutic antibodies without visiting a medical practitioner. Certain concentrations of anti-IL-23p19 antibodies are needed for pharmaceutical formulations so that the antibody can be delivered SC, IP and/or IM to the patient. These pharmaceutical formulations with a certain concentration of the anti-IL-23p19 antibody must maintain physical and chemical stability of the anti-IL-23p19 antibody. However, formulating therapeutic antibodies into aqueous pharmaceutical formulations suitable for SC, IM and/or IP administration is both challenging and unpredictable.

The challenge and unpredictability associated with formulating therapeutic antibodies into aqueous pharmaceutical formulations suitable for SC, IM and/or IP administration is due, in part, to the numerous properties a pharmaceutical formulation must possess to be therapeutically viable. Pharmaceutical formulations must provide stability to the therapeutic antibody in solution while, at the same time, maintaining the therapeutic antibody's functional characteristics essential for therapeutic efficacy such as target affinity, selectivity and potency. In addition, the aqueous pharmaceutical formulation must also be safe for administration to, and well tolerated by, patients as well as being suitable for manufacturing and storage.

Formulating high concentrations of therapeutic antibodies is even more complex. For example, increased rates of antibody degradation, cleavage, clipping, high molecular weight aggregation, dimerization, trimerization, precipitation pH shift, turbidity, solution color change, changes in charge, isomerization, oxidation and/or deamination (all of which affect the therapeutic antibody concentration, functionality and efficacy) have been reported for formulations of highly concentrated therapeutic antibodies. Another known challenge when formulating high concentrations of therapeutic antibodies is an increase in viscosity which can negatively affect SC, IM and/or IP administration of a pharmaceutical formulation.

Mirikizumab, CAS Registry No. 1884201-71-1, is a humanized immunoglobulin (Ig) G4-variant monoclonal antibody targeting the p19 subunit of human IL-23 and is described in U.S. Pat. No. 9,023,358. Mirikizumab is being evaluated for the treatment of patients with moderate to severe plaque psoriasis, UC and CD. Mirikizumab may be administered to patients subcutaneously in a highly concentrated (75-150 mg/mL) pharmaceutical formulation. It has been found in pre-formulation studies that mirikizumab is less stable in formulations at the lower and higher pH values (pH<5.0 and pH>7.0). Mirikizumab samples formulated at high concentrations exhibited more soluble aggregates relative to samples formulated at lower concentrations as determined by SEC. Moreover, certain formulations of mirikizumab at concentrations of at least 50 mg/mL showed significant protein cryo-precipitation. Pharmaceutical formulations for certain concentrations of anti-IL-23p19 antibodies are needed that avoid these observed problems. The pharmaceutical formulations provided herein satisfy the aforementioned needs. More particularly, the pharmaceutical formulations provided herein are suitable for SC, IM and/or IP administration of high concentrations of mirikizumab while preserving the functional characteristics of mirikizumab essential for therapeutic efficacy.

Accordingly, there is provided a pharmaceutical formulation comprising:
(i) 50 mg/mL-150 mg/mL of a IL-23p19 antibody;
(ii) 8 mM-12 mM of a citrate buffer;
(iii) 100-200 mM of sodium chloride (NaCl); and
(iv) 0.01% w/v to 0.05% w/v of a surfactant,
wherein the pH of the formulation is between 5.0 to 6.0,
and wherein the anti-IL-23p19 antibody comprises a light chain variable region (LCVR) and a heavy chain variable region (HCVR), the amino acid sequence of the LCVR is SEQ ID NO: 8 and the amino acid sequence of the HCVR is SEQ ID NO: 7.

In an embodiment of the present invention, the anti-IL-23p19 antibody comprises a light chain (LC) and a heavy chain (HC), wherein the amino acid sequence of the LC is SEQ ID NO: 10 and the amino acid sequence of the heavy chain is SEQ ID NO: 9.

In a preferred embodiment of the present invention, the anti-IL-23p19 antibody is mirikizumab.

In an alternative embodiment of the present invention, the pharmaceutical formulation comprises an anti-IL-23p19 antibody wherein the anti-IL-23p19 antibody comprises a LCVR and a HCVR, wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In a further embodiment of the present invention, the concentration of the anti-IL-23p19 antibody is about 75 mg/mL to about 150 mg/mL. Preferably, the concentration of the anti-IL-23p19 antibody is about 100 mg/mL to about 150 mg/mL. Further preferably, the concentration of the anti-IL-23p19 antibody is about 100 mg/mL. Alternatively, preferably, the concentration of the anti-IL-23p19 antibody is about 125 mg/mL.

In a still further embodiment of the present invention, the concentration of the citrate buffer is about 10 mM. Preferably, the citrate buffer is a sodium citrate buffer.

In a still further embodiment of the present invention, the surfactant is polysorbate 20 or polysorbate 80. Preferably, the surfactant is polysorbate 80. Further preferably, the concentration of the surfactant is about 0.03% (w/v).

In a still further embodiment of the present invention, the concentration of NaCl is about 150 mM.

In a still further embodiment of the present invention the pH of the formulation is about 5.5.

In a preferred embodiment of the present invention, the formulation comprises:
(i) 100 mg/mL or 125 mg/mL of mirikizumab;
(ii) 10 mM of sodium citrate buffer;
(iii) 150 mM of NaCl; and
(iv) 0.03% w/v of polysorbate 80,
wherein the pH of the formulation is about 5.5.

Preferably, the formulation comprises 100 mg/mL of mirikizumab.

Alternatively, preferably, the formulation comprises 125 mg/mL of mirikizumab.

In a further aspect of the present invention, there is also provided a method of treating and/or preventing psoriasis, ulcerative colitis, Crohn's Disease, psoriatic arthritis and/or ankylosing spondylitis, wherein the method comprises administering to a patient a therapeutically effective amount of a pharmaceutical formulation of the present invention.

In a still further aspect of the present invention, there is provided a pharmaceutical formulation of the present invention for use in the treatment and/or prevention of psoriasis, ulcerative colitis, Crohn's Disease, psoriatic arthritis and/or ankylosing spondylitis.

In a still further aspect of the present invention, there is provided the use of a pharmaceutical formulation of the present invention in the manufacture of a medicament for use in the treatment of psoriasis, ulcerative colitis, Crohn's Disease, psoriatic arthritis and/or ankylosing spondylitis.

In addition to the difficulties in formulating antibody therapeutics described above, undesirable injection-associated pain, even after a syringe needle is removed, has been reported with such routes of administration and can impair patient compliance with therapy. Injection-associated pain has been reported with formulations having increased viscosity. Injection-associated pain of pharmaceutical formulations comprising therapeutic antibodies is a complex, multifactorial issue. For example, each individual component, and/or concentration, ratio and characteristic thereof, of an aqueous pharmaceutical formulation can impact injection-associated pain associated with a therapeutic. Likewise, individual components (and/or concentrations, ratios and characteristics thereof) can impact the stability, functional characteristics, manufacturability and/or tolerability of a formulated therapeutic antibody in an aqueous pharmaceutical formulation. Thus, while a specific formulation adjustment may provide a beneficial impact to a given aspect of the formulation, the same adjustment may also negatively impact other aspects of the formulation. Even further adding to the complexity, a nearly limitless number of different formulation components (e.g., buffers and excipients), as well as concentrations and ratios thereof, have been reported. However, there remains little-to-no correlation for predicting the impact of a specific formulation on the various properties and characteristics of a given therapeutic antibody.

Accordingly, there is also a need for a pharmaceutical formulation of therapeutic antibodies suitable for SC, IM and/or IP administration and which is well tolerated by patients, exhibiting a therapeutically beneficial level of injection-associated pain. Even more particularly, there is a need for a pharmaceutical formulation of mirikizumab suitable for SC, IM and/or IP administration and which is well tolerated by patients, exhibiting an improved level of injection-associated pain over alternative formulations of mirikizumab. Such pharmaceutical formulation must also provide stability for the therapeutic antibody and preserve the properties of the therapeutic antibody essential for therapeutic efficacy. Such pharmaceutical formulations must also be amenable to manufacturing, preferably having an extended shelf life. Such pharmaceutical formulations must also be suitable for SC, IM and/or IP administration via a pre-filled syringe or an autoinjector.

The pharmaceutical formulations provided herein satisfy the aforementioned needs. More particularly, the pharmaceutical formulations provided herein are suitable for SC, IM and/or IP administration of high concentrations of mirikizumab (for example, appropriate viscosity) while preserving the functional characteristics of mirikizumab essential for therapeutic efficacy. The pharmaceutical formulations provided herein are also well tolerated by patients, and may exhibit an improved level of injection-associated pain over alternative pharmaceutical formulations of mirikizumab and providing a therapeutically favorable level of injection-associated pain.

Accordingly, there is provided a pharmaceutical formulation comprising:
(i) 50 mg/mL-150 mg/mL of an IL-23p19 antibody;
(ii) 3 mM-12 mM of a histidine buffer;
(iii) 25-75 mM of NaCl;
(iv) 2-5% w/v of a tonicity agent; and
(iv) 0.01% w/v to 0.05% w/v of a surfactant,
wherein the pH of the formulation is between 5.0 to 6.0,
and wherein the anti-IL-23p19 antibody comprises a light chain variable region (LCVR) and a heavy chain variable region (HCVR), the amino acid sequence of the LCVR is SEQ ID NO: 8 and the amino acid sequence of the HCVR is SEQ ID NO: 7.

In an embodiment of the present invention, the anti-IL-23p19 antibody comprises a light chain (LC) and a heavy chain (HC), wherein the amino acid sequence of the LC is SEQ ID NO: 10 and the amino acid sequence of the heavy chain is SEQ ID NO: 9.

In a preferred embodiment of the present invention, the anti-IL-23p19 antibody is mirikizumab.

In an alternative embodiment of the present invention, the pharmaceutical formulation comprises an anti-IL-23p19 antibody wherein the anti-IL-23p19 antibody comprises a LCVR and a HCVR, wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In a further embodiment of the present invention, the concentration of the anti-IL-23p19 antibody is about 75 mg/mL to about 150 mg/mL. Preferably, the concentration of the anti-IL-23p19 antibody is about 100 mg/mL to about 150 mg/mL. Further preferably, the concentration of the anti-IL-23p19 antibody is about 100 mg/mL. Alternatively, preferably, the concentration of the anti-IL-23p19 antibody is about 125 mg/mL.

In a still further embodiment of the present invention, the concentration of the histidine buffer is about 5 mM.

In a still further embodiment of the present invention, the tonicity agent is mannitol.

Preferably, the concentration of mannitol is 3.3% w/v.

In a still further embodiment of the present invention, the surfactant is polysorbate 20 or polysorbate 80.

Preferably, the surfactant is polysorbate 80.

Further preferably, the concentration of the surfactant is about 0.03% (w/v).

In a still further embodiment of the present invention, the concentration of NaCl is about 50 mM.

In a still further embodiment of the present invention, the pH of the formulation is about 5.5.

In a preferred embodiment of the present invention, the formulation comprises:
(i) 100 mg/mL or 125 mg/mL of mirikizumab;
(ii) 5 mM of a histidine buffer;
(iii) 50 mM of NaCl;
(iv) 3.3% w/v of mannitol; and
(v) 0.03% w/v of polysorbate 80,
wherein the pH of the formulation is 5.5.

Preferably, the formulation comprises 100 mg/mL of mirikizumab. Alternatively, preferably, the formulation comprises 125 mg/mL of mirikizumab.

In a further aspect of the present invention, there is provided a method of treating and/or preventing psoriasis, ulcerative colitis, Crohn's Disease, psoriatic arthritis and/or ankylosing spondylitis, wherein the method comprises administering to a patient a therapeutically effective amount of a pharmaceutical formulation of the present invention.

In a still further aspect of the present invention, there is provided a pharmaceutical formulation of the present invention for use in the treatment and/or prevention of psoriasis, ulcerative colitis, Crohn's Disease, psoriatic arthritis and/or ankylosing spondylitis.

In a still further aspect of the present invention, there is provided the use of a pharmaceutical formulation of the present invention in the manufacture of a medicament for use in the treatment of psoriasis, ulcerative colitis, Crohn's Disease, psoriatic arthritis and/or ankylosing spondylitis.

In a still further aspect of the present invention, there is provided a method of reducing injection-associated pain experienced by a patient at the time of, or shortly after, SC, IP and/or IM administration of a pharmaceutical formulation comprising an anti-IL-23p19 antibody, the method comprising administering to a patient a pharmaceutical formulation of the present invention, wherein, said step of administering provides a therapeutically favorable level of injection-associated pain.

Preferably, the therapeutically favorable level of injection-associated pain comprises a VAS score of less than 30 mm or less than 20 mm.

In a still further aspect of the present invention, there is provided an improved method for SC administration of an anti-IL-23p19 antibody to a patient in need thereof, wherein the improvement comprises a reduction in injection-associated pain upon SC administration of a pharmaceutical formulation comprising an anti-IL-23p19 antibody, the method comprising administering a pharmaceutical formulation of the present invention, wherein said step of administering provides an improved level of injection-associated pain and/or provides a therapeutically favorable level of injection-associated pain. Preferably, the therapeutically favorable level of injection-associated pain comprises a VAS score of less than 30 mm or less than 20 mm.

In a still further aspect of the present invention, there is provided an improved method of treating at least one of psoriasis, ulcerative colitis, Crohn's Disease, psoriatic arthritis and ankylosing spondylitis, wherein the improvement comprises a reduction in injection-associated pain upon the SC administration of a pharmaceutical formulation comprising an anti-IL-23p19 antibody, the method comprising administering a pharmaceutical formulation as described herein, wherein said step of administering provides an improved level of injection-associated pain and/or provides a therapeutically favorable level of injection-associated pain. Preferably, the therapeutically favorable level of injection-associated pain comprises a VAS score of less than 30 mm or less than 20 mm.

As used herein, the expression “pharmaceutical formulation” means a solution solution having at least one therapeutic antibody capable of exerting a biological effect in a human, at least one inactive ingredient (e.g., buffer, excipient, surfactant, etc.) which, when combined with the therapeutic antibody, is suitable for therapeutic administration to a human. Pharmaceutical formulations of the present disclosure are stable formulations wherein the degree of degradation, modification, aggregation, loss of biological activity and the like, of therapeutic antibodies therein, is acceptably controlled and does not increase unacceptably with time.

As used herein, the term “antibody” refers to an immunoglobulin G (IgG) molecule comprising two heavy chains (“HC”) and two light chains (“LC”) inter-connected by disulfide bonds. Each heavy chain is comprised of a heavy chain variable region (“HCVR”) and a heavy chain constant region (“CH”). Each light chain is comprised of a light chain variable region (“LCVR”) and a light chain constant region (“CL”). Each HCVR and LCVR are further sub-dividable into regions of hypervariability, termed complementarity determining regions (“CDR”), interspersed with regions that are more conserved, termed framework regions (“FR”). Each HCVR and LCVR is composed of three CDRs and four FRs arranged from amino-terminus to carboxy-terminus in the following order: FR1, CDR1, FR2, CDR2, FR3, CDR3, FR4. The variable regions of each HC and LC contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used interchangeably herein “an antibody that binds to the p19 subunit of human IL-23” or “an anti-IL-23p19 antibody” refers to an antibody that binds to the p19 subunit of human IL-23 but does not bind to the p40 subunit of human IL-23. Examples of such antibodies include mirikizumab, guselkumab, tildrakizumab and risankizumab.

Guselkumab, CAS Registry No. 1350289-85-8, is a fully human IgG1 lambda monoclonal antibody that binds to the p19 subunit of human IL-23 that has been approved for the treatment of plaque psoriasis. The antibody and methods of making same are described in U.S. Pat. No. 7,935,344.

Tildrakizumab, CAS Registry No. 1326244-10-3, is a humanized, IgG1 kappa monoclonal antibody targeting the p19 subunit of human IL-23 that has approved for the treatment of moderate to severe plaque psoriasis. The antibody and methods of making same are described in U.S. Pat. No. 8,293,883.

Risankizumab, CAS Registry No. 1612838-76-2, is a humanized, IgG1 kappa monoclonal antibody targeting the p19 subunit of human IL-23. The antibody and methods of making same are described in U.S. Pat. No. 8,778,346. Risankizumab is has been approved for the treatment moderate to severe plaque psoriasis.

Brazikumab, CAS Registry No. 1610353-18-8, is a humanized, IgG2-lambda monoclonal antibody targeting the p19 subunit of human IL-23. The antibody and methods of making same are described in U.S. Pat. No. 8,722,033. Brazikumab is being evaluated for the treatment CD and UC.

As may be used herein, the terms “about” or “approximately”, when used in reference to a particular recited numerical value or range of values, means that the value may vary from the recited value by no more than 10% (e.g., +/−10%). For example, as used herein, the expression “about 100” includes 90 and 110 and all values in between (e.g., 91, 92, 93, 94, etc.).

As used herein, the phrase “injection site pain” refers to pain attributable to injection of a liquid formulation subcutaneously and localized to the site of the injection. Pain may be evaluated using any type of pain assessment known in the art, including, for example, visual analog scales (VAS), qualitative assessments of pain, or needle pain assessments. For example, subject-perceived injection site pain may be assessed using the Pain Visual Analog Scale (VAS). A VAS is a measurement instrument that measures pain as it ranges across a continuum of values, e.g., from none to an extreme amount of pain. Operationally, a VAS is a horizontal line, about 100 mm in length, anchored by numerical and/or word descriptors, e.g., 0 or 10, or “no pain” or “excruciating pain,” optionally with additional word or numeric descriptors between the extremes, e.g., mild, moderate, and severe; or 1 through 9) (see, e.g., Lee J S, et al. (2000) AcadEmerg Med 7:550, or Singer and Thods (1998) Academic Emergency Medicine, 5:1007). Pain may be assessed at a single time or at various times following administration of a formulation such as, for example, immediately after injection, at about 1, 2, 3, 4, 5, 6, 7, 8, 9, 10, 15, 20, 25, 30, 35, 40, or 45 minutes after injection. Severity of pain may be categorized, according to the VAS tool, as mild pain (≤30 mm); moderate pain (>30 mm-≤70 mm) and severe pain (>70 mm). A desired property of a stable pharmaceutical formulation is being well tolerated by patients, for example, providing a therapeutically favorable level of injection-associated pain (e.g., a VAS score of <30 mm and/or <20 mm). As is known, the components, and concentrations and/or ratios thereof, of a pharmaceutical formulation may impact injection-associated pain experienced by the patient.

As used interchangeably herein, “treatment” and/or “treating” and/or “treat” are intended to refer to all processes wherein there may be a total elimination, slowing or delaying, reduction in severity or frequency (e.g., of flares or episodes), interruption or stopping of the progression of disease and/or symptoms thereof, but does not require a total elimination of all disease symptoms. Treatment includes administration of an aqueous pharmaceutical formulation of the present disclosure for treatment of a disease in a human that would benefit from at least one of the above-listed processes, including: (a) inhibiting further progression of disease symptoms and effects, i.e., arresting its development; (b) relieving the disease, i.e., causing an elimination or regression of disease, disease symptoms or complications thereof; and (c) preventing or reducing the frequency of disease episodes or flares. According to specific embodiments, the pharmaceutical formulations provided herein may be used in the treatment of at least one of psoriasis, ulcerative colitis, Crohn's Disease, psoriatic arthritis and/or ankylosing spondylitis.

As used interchangeably herein, the term “patient,” “subject” and “individual,” refers to a human. Unless otherwise noted, the subject is further characterized as having, being at risk of developing, or experiencing symptoms of a disease that would benefit from administration of a pharmaceutical formulation disclosed herein.

As used interchangeably herein, an “effective amount” or “therapeutically effective amount” of a pharmaceutical formulation of the instant disclosure refers to an amount necessary (at dosages, frequency of administration and for periods of time for a particular means of administration) to achieve the desired therapeutic result. An effective amount of pharmaceutical formulation of the present disclosure may vary according to factors such as the disease state, age, sex, and weight of the subject and the ability of the pharmaceutical formulation of the present disclosure to elicit a desired response in the subject. An effective amount is also one in which any toxic or detrimental effects of the pharmaceutical formulation of the present disclosure are outweighed by the therapeutically beneficial effects.

The pharmaceutical formulations of the present invention may be administered to a patient via parenteral administration. Parenteral administration, as understood in the medical field, refers to the injection of a dose into the body by a sterile syringe or some other drug delivery system including an autoinjector or an infusion pump. Exemplary drug delivery systems for use with the pharmaceutical formulations of the present disclosure are described in the following references, the disclosures of which are expressly incorporated herein by reference in their entirety: U.S. Patent Publication No. 2014/0054883 to Lanigan et al., filed Mar. 7, 2013 and entitled “Infusion Pump Assembly”; U.S. Pat. No. 7,291,132 to DeRuntz et al., filed Feb. 3, 2006 and entitled “Medication Dispensing Apparatus with Triple Screw Threads for Mechanical Advantage”; U.S. Pat. No. 7,517,334 to Jacobs et al., filed Sep. 18, 2006 and entitled “Medication Dispensing Apparatus with Spring-Driven Locking Feature Enabled by Administration of Final Dose”; and U.S. Pat. No. 8,734,394 to Adams et al., filed Aug. 24, 2012 and entitled “Automatic Injection Device with Delay Mechanism Including Dual Functioning Biasing Member.” Parenteral routes include IM, SC and IP routes of administration.

### BRIEF DESCRIPTION OF FIGURES

FIG. 1 is a contour plot of mirikizumab concentration vs. pH that shows the relationship of target pH to antibody concentration on predicted monomer purity.

FIG. 2 illustrates the glide force data for Formulations 1 and 21-29.

### EXAMPLES

### Example 1: Production of Antibodies

Anti-IL-23p19 antibodies can be made and purified as follows. An appropriate host cell, such as CHO, is either transiently or stably transfected with an expression system for secreting antibodies using an optimal predetermined HC:LC vector ratio or a single vector system encoding both LC and both HC, such as each LC being SEQ ID NO: 10 and each HC being SEQ ID NO: 9. Clarified media, into which the antibody has been secreted, is purified using any of many commonly-used techniques. For example, the medium may be conveniently applied to a Protein A or G Sepharose FF column that has been equilibrated with a compatible buffer, such as phosphate buffered saline (pH 7.4). The column is washed to remove nonspecific binding components. The bound antibody is eluted, for example, by pH gradient. Antibody fractions are detected, such as by SDS-PAGE, and then are pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, or hydroxyapatite chromatography. The purity of the antibody after these chromatography steps is greater than 99%. The product may be immediately frozen at −70° C. in the formulation matrix of the invention or may be lyophilized. The amino acid and nucleic acid sequences for the exemplified antibody are provided below.

### Example 2: Formulation Study A

### Study Design and Preparation of Anti-IL-23p19 Antibody Pharmaceutical Formulations

The study design assessed the impact of four factors: concentration of anti-IL-23p19 antibody (mirikizumab), concentration of sodium chloride, concentration of polysorbate 80 and pH. The formulations assessed are shown in Table 1.

TABLE 1
Study design

| | | Antibody Conc. | Ps-80 | NaCl | |
|---|---|---|---|---|---|
| Formulation | pH | (mg/mL) | (% w/v) | (mM) | Container |
| 1 | 5.5 | 85 | 0.03 | 150 | PFS (1 mL) |
| 2 | 5.5 | 20 | 0.01 | 100 | PFS (1 mL) |
| 3 | 5.0 | 20 | 0.01 | 200 | PFS (1 mL) |
| 4 | 6.0 | 150 | 0.05 | 100 | PFS (1 mL) |
| 5 | 6.0 | 20 | 0.05 | 200 | PFS (1 mL) |
| 6 | 6.0 | 85 | 0.01 | 200 | PFS (1 mL) |
| 7 | 6.0 | 150 | 0.01 | 150 | PFS (1 mL) |
| 8 | 5.0 | 20 | 0.05 | 150 | PFS (1 mL) |
| 9 | 5.0 | 85 | 0.05 | 100 | PFS (1 mL) |
| 10 | 5.5 | 150 | 0.05 | 200 | PFS (1 mL) |
| 11 | 5.0 | 150 | 0.03 | 200 | PFS (1 mL) |
| 12 | 6.0 | 20 | 0.03 | 100 | PFS (1 mL) |
| 13 | 5.0 | 150 | 0.01 | 100 | PFS (1 mL) |
| 14 | 5.5 | 85 | 0.03 | 150 | PFS (1 mL) |
| 15 | 5.5 | 100 | 0.03 | 150 | PFS (1 mL) |
| 16 | 5.5 | 125 | 0.03 | 150 | PFS (1 mL) |
| 17 | 5.5 | 50 | 0.05 | 150 | Vial |
| 18 | 5.5 | 20 | 0.01 | 150 | Vial |
| 19 | 5.5 | 20 | 0.05 | 150 | Vial |
| 20 | 5.5 | 125 | 0.03 | 150 | PFS (2 mL) |

The antibody concentration was examined in Formulations 1-20 at 20, 85, 100, 125 and 150 mg/mL. The wide antibody concentration was chosen to account for multiple possible presentations for mirikizumab drug product and based on pre-formulation data which provided clear correlations between some forms of degradation (such as aggregation) and concentration. Polysorbate 80 was studied at three concentrations (0.01, 0.03 and 0.05% w/v). NaCl effects were explored at the concentrations 100, 150 and 200 mM. pH effects were studied over 5.0 to 6.0 as pre-formulation studies and biophysical screening indicated that the regional of optimal global stability was pH 5.5 to 6.0.

Based on pre-formulation data, no significant effects on stability were observed from various container closure types. Therefore, a 1 mL prefilled syringe (PFS) was used to cover the study design for consistency. Vials were used for Formulations 17-19. Formulation 20 (with a 2 mL PFS) was included as a direct comparison with Formulation 16 to determine if there may be a significant contribution from different syringes.

Formulations 1-20 were independently prepared in the order specified. The material for each formulation was prepared by dialyzing drug substance into the specified formulation condition. Dialyzed solution was then spiked with an appropriate amount of polysorbate and diluted to the prescribed antibody concentration with formulation buffer. Samples were filtered with 0.22 μm filters and aseptically filled into the designated container closure systems.

The buffer excipient composition consists of citric acid anhydrous (QD514N, Lot No. C490136), sodium citrate dihydrate (QD517A, Lot No. C487212), sodium chloride (QD515R, Lot No. C481616), polysorbate 80 (QD513DVIE, Lot No. C457300).

The anti-IL-23p19 antibody is mirikizumab, which comprises a LC of SEQ ID NO: 10, and a HC of SEQ ID NO: 9 (Demo Lot No. EL01685-039-F-Fill).

Analytical and characterization techniques selected to measure the chemical and physical stability and properties of the formulations included size exclusion chromatography (SEC) HPLC, imaged capillary isoeletric focusing iCIEF, reduced and non-reduced CESDS, HIAC, microflow imaging (MFI), visual appearance, pH (USP <921>), UV absorbance to measure protein concentration syringe functionality and device testing.

Samples were stored at four temperature conditions (5° C., 15° C., 25° C. and 35° C.) with the syringe stored horizontally and vials inverted. This range of temperatures enables estimations of the activation energies of each analytical response variable assuming Arrhenius kinetics. In addition, higher temperature storage enabled earlier prediction of optimal formulation conditions to speed the drug product development process.

The sampling schedule for Formulations 1-14 is outlined in Table 2. The schedule is designed to capture four time points for 25° C. and 35° C. at three months and three time points for other storage conditions. This sampling frequency permits sufficient information to fit the data in prediction models. After the three-month time point, activation energies (Ea) were calculated employing an Arrhenius kinetic model to correlate results at accelerated temperatures with predicted 5° C. stability. An Ea value of 21.5 kcal/mol was used to fit the SEC (monomer, polymer and post-monomer), iCIEF (main peak, total acidic and total basic variants), and non-reduced and reduced CE-SDS. This fit is based in part upon what has been observed with other IgG4 antibodies. The time points denoted by X are conditions where samples were analyzed by SEC, iCIEF, reduced and non-reduced CE-SDS, pH, UV content and visual appearance. Testing by HIAC and MFI was performed less frequently.

TABLE 2
Sampling schedule for Formulations 1-14

| Temp | | Weeks | Months | | | |
|---|---|---|---|---|---|---|
| (° C.) | Initial | 2 | 1 | 2 | 3 | 6 |
| 5 | X^{a, b, c} | | | X | X^{a, b} | X^{a, b} |
| 15 | | | | X | X^{b} | |
| 25 | | | X^{b} | X | X^{a, b} | X^{a, b} |
| 35 | | | X^{b} | X | X^{a, b} | |

X = sample time point, ^{a}HIAC sample, ^{b}MFI sample.

The sampling schedule for Formulations 15-20 is shown in Table 3. Formulations 15-20 represent formulations may be assessed in clinical trials in human patients. These formulations were put in relevant container closure systems (which included vials and the 2.25 mL syringes). These formulations were assessed to confirm stability of these potential drug products and to understand if there were any effects of container closure type on stability.

TABLE 3
Sampling schedule for Formulations 15-20

| Temp | | Weeks | Months | | | | | | |
|---|---|---|---|---|---|---|---|---|---|
| (° C.) | Initial | 2 | 1 | 2 | 3 | 6 | 9 | 12 | 18 |
| 5 | X^{a, b, c} | | | | X^{a, b} | X^{b} | X | X^{a, b} | X^{b} |
| 15 | | | | | | | | | |
| 25 | | | X^{b} | | X^{a, b} | X | | | |
| 35 | | X | X^{b} | X | X^{a, b} | | | | |

X = sample time point, ^{a}HIAC sample, ^{b}MFI sample.

### Formulation Study A—Results—Size Exclusion Chromatography

SEC percent monomer values at 5° C., 15° C., 25° C. and 35° C. are shown in Tables 4a-4d. The 35° C. data are displayed through three months. The 25° C. data are displayed through 6 months, and 5° C. data are shown up to 18 months (only for Formulations 15 and 20). Increasing temperature resulted in decreases in percent monomer. The largest changes in this data set are <2%. Percent monomer is remains above 98.6% for samples tested at 5° C. through 18 months except for one result at 9 months.

Monomer and polymer values (not shown) inversely mirror each other closely and degradation observed by SEC was primarily the result of soluble aggregate (polymer) formation.

Predicted effects of each input variable on SEC monomer purity over 24-months at 5° C. are modelled using results obtained from data up to 3 months. All four temperatures were used to model the modified Arrhenius kinetics. An activation energy (Ea) of 21.5 kcal/mol was used to generate these predictions. Predictions of percent monomer in all cases are >98% and the largest predicted change is >1.3% indicating that mirikizumab is stable over the entire design region. This is in close agreement with the empirical data shown in Tables 4a-4d. Increased mirikizumab concentration through the range studied resulted in greater monomer loss. This relationship is likely a function of the increased probability of intermolecular interactions between antibodies. Slightly increased stability was observed at lower pH conditions in the study consistent with preformation studies. Polysorbate 80 concentration, NaCl concentration and container closure appear to have no significant effect. For the two factors that had an effect (antibody concentration, pH) the difference between the best and worst locations in the design region was <1.0%.

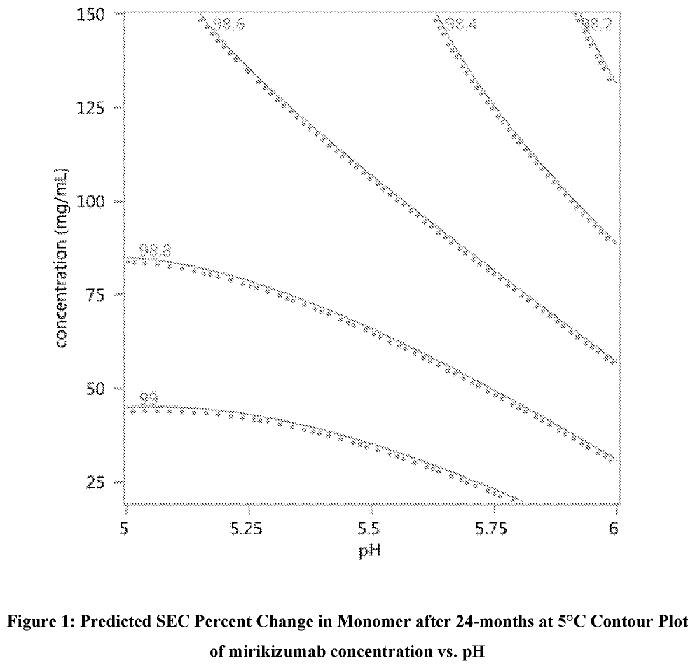

FIG. 1 is a contour plot that shows the relationship of target pH to antibody concentration on predicted monomer purity. The Prob>F Effect Test value for pH Target*Concentration Target is 0.0130 indicating that interaction is statistically significant. The pH effect on purity is stronger at higher antibody concentration.

Table 4a: SEC Percent Monomer at 5° C.

| Formu- | | | | | | | |
|---|---|---|---|---|---|---|---|
| lation | | | | | | | |
| No. | T = 0 | 2 M | 3 M | 6 M | 9 M | 12 M | 18 M |
| 1 | 99.0355 | 98.8508 | 98.8962 | 98.9358 | | | |
| 2 | 99.2563 | 99.2886 | 99.3144 | 99.2253 | | | |
| 3 | 99.3694 | 99.3511 | 99.4345 | 99.3993 | | | |
| 4 | 98.6003 | 98.5643 | 98.5050 | 98.4485 | | | |
| 5 | 99.1434 | 99.1082 | 99.0041 | 99.0904 | | | |
| 6 | 98.7584 | 98.7591 | 98.7379 | 98.6326 | | | |
| 7 | 98.6183 | 98.2753 | 98.3667 | 98.4292 | | | |
| 8 | 99.3969 | 99.3085 | 99.4059 | 99.3078 | | | |
| 9 | 99.2110 | 99.1204 | 99.1587 | 99.0774 | | | |
| 10 | 98.8394 | 98.5198 | 98.7985 | 98.7467 | | | |
| 11 | 98.9706 | 99.0593 | 99.0224 | 98.9975 | | | |
| 12 | 99.0425 | 99.0592 | 99.0944 | 98.9633 | | | |
| 13 | 99.1072 | 98.8357 | 99.0167 | 98.9584 | | | |
| 14 | 99.0121 | 98.9175 | 98.9514 | 98.8717 | | | |
| 15 | 99.0650 | | 98.9610 | 98.9082 | 97.1087 | 98.6133 | 98.6654 |
| 16 | 99.0472 | | 98.9007 | 98.8719 | 98.6259 | 98.5634 | |
| 17 | 99.1902 | | 99.0789 | 99.0568 | 98.9996 | | |
| 18 | 99.2656 | | 99.3488 | 99.2322 | 99.0923 | 99.0771 | |
| 19 | 99.3073 | | 99.2641 | 99.2157 | 98.8992 | 99.0655 | |
| 20 | 98.9758 | | 98.9151 | 98.8217 | 98.8308 | 98.5748 | 98.6104 |

5° C.

TABLE 4b
SEC Percent Monomer at 15° C.

| Formulation | T = 0 | 2M | 3M |
|---|---|---|---|
| 1 | 99.03 | 98.64 | 98.80 |
| 2 | 99.25 | 99.16 | 99.28 |
| 3 | 99.36 | 99.34 | 99.32 |
| 4 | 98.60 | 98.25 | 98.40 |
| 5 | 99.14 | 98.91 | 99.13 |
| 6 | 98.75 | 98.57 | 98.63 |
| 7 | 98.61 | 98.24 | 98.29 |
| 8 | 99.39 | 99.21 | 99.30 |
| 9 | 99.21 | 99.02 | 99.10 |
| 10 | 98.83 | 98.71 | 98.76 |
| 11 | 98.97 | 98.93 | 98.97 |
| 12 | 99.04 | 98.93 | 99.12 |
| 13 | 99.10 | 98.84 | 98.96 |
| 14 | 99.01 | 98.83 | 98.89 |
| 15 | 99.06 | | |
| 16 | 99.04 | | |
| 17 | 99.19 | | |
| 18 | 99.26 | | |
| 19 | 99.30 | | |
| 20 | 98.97 | | |

15° C.

TABLE 4c
SEC Percent Monomer at 25° C.

| Formulation No. | T = 0 | 1M | 2M | 3M | 6M |
|---|---|---|---|---|---|
| 1 | 99.0355 | 98.7580 | 98.5574 | 98.4367 | 98.0368 |
| 2 | 99.2563 | 99.1589 | 99.1105 | 99.0405 | 98.5627 |
| 3 | 99.3694 | 99.1199 | 99.2184 | 99.2363 | 98.5666 |
| 4 | 98.6003 | 98.2757 | 98.0982 | 97.9622 | 97.6140 |
| 5 | 99.1434 | 98.8306 | 98.8633 | 98.9352 | 98.6050 |
| 6 | 98.7584 | 98.5073 | 98.3420 | 98.2210 | 97.9763 |
| 7 | 98.6183 | 98.1165 | 97.7388 | 97.8497 | 97.4158 |
| 8 | 99.3969 | 99.2152 | 98.9631 | 99.0373 | 98.6140 |
| 9 | 99.2110 | 98.9730 | 98.5146 | 98.5381 | 97.9131 |
| 10 | 98.8394 | 98.3577 | 98.3127 | 98.2688 | 97.7979 |
| 11 | 98.9706 | 98.7703 | 98.4922 | 98.3596 | 97.7233 |
| 12 | 99.0425 | 98.9297 | 98.7366 | 98.9000 | 98.4723 |
| 13 | 99.1072 | 98.8193 | 98.5467 | 98.3160 | 97.6511 |
| 14 | 99.0121 | 98.8272 | 98.5284 | 98.5095 | 97.9758 |
| 15 | 99.0650 | 98.8577 | | 98.5196 | |
| 16 | 99.0472 | 98.6574 | | 98.4515 | |
| 17 | 99.1902 | 98.8746 | | 98.7144 | |
| 18 | 99.2656 | 99.2270 | | 99.1250 | |
| 19 | 99.3073 | 99.1136 | | 99.0706 | |
| 20 | 98.9758 | 98.7749 | | 98.5271 | |

25° C.

TABLE 4d
SEC Percent Monomer at 35° C.

| Formulation No. | T = 0 | 0.5M | 1M | 2M | 3M |
|---|---|---|---|---|---|
| 1 | 99.0355 | | 98.4677 | 97.9971 | 97.7652 |
| 2 | 99.2563 | | 99.0013 | 98.7378 | 98.6375 |
| 3 | 99.3694 | | 99.1080 | 98.7451 | 98.3767 |
| 4 | 98.6003 | | 97.8095 | 97.5004 | 97.0372 |
| 5 | 99.1434 | | 98.8166 | 98.5338 | 98.5051 |
| 6 | 98.7584 | | 98.3243 | 97.9466 | 97.8319 |
| 7 | 98.6183 | | 97.8843 | 97.4690 | 97.1288 |
| 8 | 99.3969 | | 98.9599 | 98.4933 | 98.0880 |
| 9 | 99.2110 | | 98.5422 | 97.9500 | 97.4241 |
| 10 | 98.8394 | | 98.3525 | 97.4123 | 97.1977 |
| 11 | 98.9706 | | 98.3487 | 97.5550 | 97.0332 |
| 12 | 99.0425 | | 98.8261 | 98.4669 | 98.5462 |
| 13 | 99.1072 | | 98.3253 | 97.5610 | 96.9009 |
| 14 | 99.0121 | | 98.4796 | 98.0903 | 97.4965 |
| 15 | 99.0650 | 98.6582 | 98.4581 | 97.9458 | 97.4352 |
| 16 | 99.0472 | 98.7509 | 98.4086 | 97.7286 | 97.5245 |
| 17 | 99.1902 | 98.9399 | 98.6580 | | 98.0500 |
| 18 | 99.2656 | 99.1655 | 99.0648 | | 98.7695 |
| 19 | 99.3073 | 99.0714 | 99.0123 | | 98.5604 |
| 20 | 98.9758 | 98.8358 | 98.4525 | | 97.3536 |

35° C.

Formulation Study A: Results—Charge Heterogeneity—iCIEF

### a) Percent Main Peak

iCIEF percent main peak values at 5° C., 15° C., 25° C. and 35° C. are shown in Tables 5a-5d. Initial values for main peak conditions were between 76.2 and 77.9% for all of the formulations. The rate main peak degradation correlates with increasing temperature. Degradation is minimal over 18 months at 5° C. where the percent main peak remaining is above 75%.

An apparent Ea estimate of 21.5 kcal/mol was used for predictions. 24-month peak predictions at 5° C. were made for percent change as a function of the five input variables (based on data up to three months). The effects of the five input variables are largest for pH though still below a <2% difference. The only two input variables which exhibited a statistically significant effect were pH and NaCl concentrations. Increased NaCl concentration appears to result in increased main peak percent. Optimal stability for pH occurs between 5.5 and 6.0. Polysorbate 80, mirikizumab concentration and container closure display no clear effects across the region studied.

TABLE 5a
iCIEF percent main peak values at 5° C.

| Formu- | | | | | | | |
|---|---|---|---|---|---|---|---|
| lation | | | | | | | |
| No. | T = 0 | 2 M | 3 M | 6 M | 9 M | 12 M | 18 M |
| 1 | 76.249 | 76.457 | 77.4974 | 77.0197 | | | |
| 2 | 76.771 | 76.0299 | 78.0262 | 77.194 | | | |
| 3 | 77.483 | 77.5245 | 77.247 | 76.7673 | | | |
| 4 | 76.814 | 76.6595 | 77.6503 | 76.8323 | | | |
| 5 | 76.932 | 78.1101 | 77.5302 | 78.0785 | | | |
| 6 | 76.979 | 76.4634 | 77.6098 | 77.9768 | | | |
| 7 | 77.885 | 77.0024 | 76.8658 | 77.2929 | | | |
| 8 | 77.347 | 77.8472 | 76.4671 | 76.9217 | | | |
| 9 | 77.319 | 75.8231 | 77.7577 | 76.7761 | | | |
| 10 | 77.65 | 77.0708 | 77.5101 | 76.3533 | | | |
| 11 | 77.464 | 76.7833 | 76.7427 | 76.7959 | | | |
| 12 | 77.491 | 77.6075 | 77.3274 | 76.9652 | | | |
| 13 | 77.188 | 76.9076 | 77.352 | 76.3947 | | | |
| 14 | 76.358 | 78.3481 | 78.3347 | 77.7161 | | | |
| 15 | 76.857 | | 76.9503 | 76.6911 | 75.9130 | 75.1500 | 75.6132 |
| 16 | 77.479 | | 77.7609 | 77.1041 | 76.3997 | 75.3519 | |
| 17 | 76.489 | | 77.5801 | 77.4066 | 76.3635 | | |
| 18 | 77.229 | | 76.6101 | 76.7367 | 75.8246 | 75.9435 | |
| 19 | 77.164 | | 77.0661 | 76.7541 | 77.0427 | 75.8500 | |
| 20 | 76.406 | | 77.7999 | 76.865 | 76.4249 | 75.6967 | 75.7152 |

5° C.

TABLE 5b
iCIEF percent main peak values at 15° C.

| Formulation No. | T = 0 | 2M | 3M |
|---|---|---|---|
| 1 | 76.249 | 77.018 | 77.1088 |
| 2 | 76.771 | 76.7464 | 76.5012 |
| 3 | 77.483 | 77.2721 | 75.7589 |
| 4 | 76.814 | 76.8649 | 76.7558 |
| 5 | 76.932 | 76.3058 | 76.3988 |
| 6 | 76.979 | 76.9648 | 77.0176 |
| 7 | 77.885 | 76.2023 | 77.0162 |
| 8 | 77.347 | 76.8247 | 75.5478 |
| 9 | 77.319 | 75.8862 | 75.6762 |
| 10 | 77.65 | 76.1686 | 76.7645 |
| 11 | 77.464 | 76.156 | 76.8353 |
| 12 | 77.491 | 76.4543 | 77.2787 |
| 13 | 77.188 | 75.5027 | 75.9949 |
| 14 | 76.358 | 76.1096 | 76.2348 |
| 15 | 76.857 | | |
| 16 | 77.479 | | |
| 17 | 76.489 | | |
| 18 | 77.229 | | |
| 19 | 77.164 | | |
| 20 | 76.406 | | |

15° C.

TABLE 5c
iCIEF percent main peak values at 25° C.

| Formulation | T = 0 | 1M | 2M | 3M | 6M |
|---|---|---|---|---|---|
| 1 | 76.2494 | 74.667 | 72.739 | 71.281 | 64.719 |
| 2 | 76.7706 | 75.479 | 73.460 | 71.231 | 65.428 |
| 3 | 77.4833 | 73.927 | 73.182 | 69.617 | 61.755 |
| 4 | 76.8135 | 74.920 | 73.114 | 71.233 | 64.860 |
| 5 | 76.9318 | 73.989 | 74.204 | 71.919 | 65.788 |
| 6 | 76.9791 | 75.084 | 72.624 | 71.983 | 65.544 |
| 7 | 77.8846 | 74.783 | 73.530 | 71.400 | 65.457 |
| 8 | 77.3469 | 73.283 | 71.773 | 69.980 | 61.547 |
| 9 | 77.3187 | 74.263 | 70.755 | 69.818 | 61.047 |
| 10 | 77.6495 | 74.521 | 73.653 | 71.547 | 65.071 |
| 11 | 77.4639 | 74.478 | 71.287 | 70.036 | 62.868 |
| 12 | 77.4914 | 74.178 | 73.113 | 70.937 | 63.873 |
| 13 | 77.1884 | 73.483 | 72.397 | 69.136 | 61.657 |
| 14 | 76.3577 | 74.884 | 73.516 | 71.257 | 65.247 |
| 15 | 76.8571 | 74.310 | | 71.699 | |
| 16 | 77.4791 | 73.765 | | 72.229 | |
| 17 | 76.4893 | 73.936 | | 71.531 | |
| 18 | 77.2288 | 73.580 | | 71.225 | |
| 19 | 77.1640 | 73.895 | | 70.954 | |
| 20 | 76.4055 | 73.360 | | 71.461 | |

25° C.

TABLE 5d
iCIEF percent main peak values at 35° C.

| Formulation | T = 0 | 0.5M | 1M | 2M | 3M |
|---|---|---|---|---|---|
| 1 | 76.249 | | 70.814 | 64.766 | 59.624 |
| 2 | 76.770 | | 69.274 | 63.514 | 57.864 |
| 3 | 77.483 | | 67.858 | 62.934 | 55.922 |
| 4 | 76.813 | | 68.420 | 63.615 | 57.764 |
| 5 | 76.931 | | 69.400 | 64.937 | 59.456 |
| 6 | 76.979 | | 69.068 | 63.599 | 59.802 |
| 7 | 77.884 | | 69.744 | 63.988 | 58.920 |
| 8 | 77.346 | | 69.256 | 62.031 | 55.697 |
| 9 | 77.318 | | 67.767 | 59.578 | 54.613 |
| 10 | 77.649 | | 69.707 | 63.348 | 59.378 |
| 11 | 77.463 | | 69.043 | 60.957 | 55.869 |
| 12 | 77.491 | | 68.614 | 61.646 | 56.415 |
| 13 | 77.188 | | 68.923 | 60.141 | 55.810 |
| 14 | 76.357 | | 70.403 | 62.865 | 58.354 |
| 15 | 76.857 | 74.420 | 69.666 | 63.888 | 58.550 |
| 16 | 77.479 | 74.104 | 69.457 | 63.532 | 58.602 |
| 17 | 76.489 | 75.532 | 69.023 | | 58.257 |
| 18 | 77.228 | 75.347 | 69.786 | | 57.887 |
| 19 | 77.164 | 75.497 | 68.591 | | 58.091 |
| 20 | 76.405 | 75.680 | 69.207 | | 58.166 |

35° C.

### b) Acidic and Basic Variants

Total acidic variants values at 5° C., 15° C., 25° C. and 35° C. are shown in Tables 6a-6d. Total basic variants values at 5° C., 15° C., 25° C. and 35° C. are shown in Tables 6e-6h.

Acidic variants increased over the course of the 18 months of data collected while only very small changes in basic variants over time were observed, except at 35° C. Acidic variants trends mirror main peak behaviour with increasing temperature causing increased acidic variant formation. Acidic variants likely arise primarily from deamidation.

Similar to the data for the main peak, the effects of all the input variables on 24-month change predictions for acidic variants and basic variants are <1%. The largest effect is derived from pH but the trends are different between the two variant forms. Acidic variants appears more stable closer to pH 5.5 while percent basic variants is most stable at pH 6.0. These two distinct trends combine to result in pH environments between pH 5.5 and 6.0 being the most chemically stable for the antibody.

TABLE 6a
Total acidic variants at 5° C.

| Formu- | | | | | | | |
|---|---|---|---|---|---|---|---|
| lation | | | | | | | |
| No. | T = 0 | 2 M | 3 M | 6 M | 9 M | 12 M | 18 M |
| 1 | 20.405 | 21.631 | 20.365 | 20.305 | | | |
| 2 | 20.144 | 22.229 | 19.786 | 20.232 | | | |
| 3 | 19.823 | 20.694 | 20.741 | 20.415 | | | |
| 4 | 20.054 | 21.594 | 20.493 | 20.567 | | | |
| 5 | 20.410 | 20.203 | 20.602 | 19.764 | | | |
| 6 | 19.907 | 21.888 | 20.419 | 19.607 | | | |
| 7 | 19.794 | 21.074 | 21.186 | 20.043 | | | |
| 8 | 19.974 | 20.386 | 21.496 | 20.425 | | | |
| 9 | 19.894 | 22.175 | 20.200 | 20.288 | | | |
| 10 | 19.668 | 21.048 | 20.368 | 20.27 | | | |
| 11 | 19.839 | 21.291 | 21.262 | 20.505 | | | |
| 12 | 19.773 | 20.771 | 20.546 | 20.235 | | | |
| 13 | 19.618 | 21.076 | 20.544 | 20.656 | | | |
| 14 | 20.198 | 19.973 | 19.742 | 19.739 | | | |
| 15 | 19.948 | | 21.106 | 20.615 | 20.979 | 21.717 | 21.457 |
| 16 | 19.882 | | 20.174 | 20.238 | 20.637 | 21.203 | |
| 17 | 19.975 | | 20.428 | 19.920 | 20.631 | | |
| 18 | 20.009 | | 21.487 | 20.575 | 21.214 | 21.128 | |
| 19 | 20.087 | | 21.065 | 20.764 | 20.113 | 21.097 | |
| 20 | 20.239 | | 20.195 | 20.329 | 20.698 | 21.225 | 21.503 |

5° C.

TABLE 6b
Total acidic variants at 15° C.

| Formulation | T = 0 | 2M | 3M |
|---|---|---|---|
| 1 | 20.405 | 21.094 | 20.703 |
| 2 | 20.144 | 21.452 | 21.256 |
| 3 | 19.823 | 20.816 | 21.849 |
| 4 | 20.054 | 21.384 | 21.375 |
| 5 | 20.410 | 21.836 | 21.688 |
| 6 | 19.907 | 21.282 | 20.841 |
| 7 | 19.794 | 21.696 | 20.831 |
| 8 | 19.974 | 21.303 | 22.099 |
| 9 | 19.894 | 22.005 | 21.858 |
| 10 | 19.668 | 21.885 | 21.028 |
| 11 | 19.839 | 21.815 | 20.739 |
| 12 | 19.773 | 21.756 | 20.773 |
| 13 | 19.618 | 22.337 | 21.732 |
| 14 | 20.198 | 21.934 | 21.667 |
| 15 | 19.948 | | |
| 16 | 19.882 | | |
| 17 | 19.975 | | |
| 18 | 20.009 | | |
| 19 | 20.087 | | |
| 20 | 20.239 | | |

15° C.

TABLE 6c
Total acidic variants at 25° C.

| Formulation | | | | | |
|---|---|---|---|---|---|
| No. | T = 0 | 1 M | 2 M | 3 M | 6 M |
| 1 | 20.405 | 23.154 | 25.009 | 25.802 | 31.253 |
| 2 | 20.144 | 22.428 | 24.200 | 25.931 | 31.260 |
| 3 | 19.823 | 23.402 | 24.232 | 26.853 | 33.596 |
| 4 | 20.054 | 22.898 | 24.786 | 26.447 | 32.815 |
| 5 | 20.410 | 23.706 | 23.756 | 25.676 | 31.688 |
| 6 | 19.907 | 22.897 | 25.296 | 25.666 | 31.931 |
| 7 | 19.794 | 23.153 | 24.269 | 26.235 | 32.097 |
| 8 | 19.974 | 23.985 | 25.571 | 26.640 | 33.821 |
| 9 | 19.894 | 23.270 | 26.540 | 26.829 | 34.104 |
| 10 | 19.668 | 23.144 | 23.999 | 25.518 | 30.914 |
| 11 | 19.839 | 23.084 | 25.823 | 26.541 | 32.427 |
| 12 | 19.773 | 23.823 | 24.900 | 26.561 | 33.665 |
| 13 | 19.618 | 24.277 | 25.025 | 26.850 | 34.023 |
| 14 | 20.198 | 22.963 | 24.121 | 25.414 | 31.347 |
| 15 | 19.948 | 23.551 | | 25.075 | |
| 16 | 19.882 | 24.070 | | 24.612 | |
| 17 | 19.975 | 23.823 | | 25.238 | |
| 18 | 20.009 | 23.993 | | 25.571 | |
| 19 | 20.087 | 23.784 | | 25.907 | |
| 20 | 20.239 | 24.615 | | 25.401 | |

25° C.

TABLE 6d
Total acidic variants at 35° C.

| Formulation No. | T = 0 | 0.5M | 1M | 2M | 3M |
|---|---|---|---|---|---|
| 1 | 20.405 | | 26.308 | 32.259 | 36.581 |
| 2 | 20.144 | | 27.779 | 33.565 | 38.232 |
| 3 | 19.823 | | 28.542 | 33.127 | 37.304 |
| 4 | 20.054 | | 29.163 | 34.172 | 40.030 |
| 5 | 20.410 | | 28.107 | 32.888 | 38.178 |
| 6 | 19.907 | | 28.655 | 34.198 | 37.723 |
| 7 | 19.794 | | 27.991 | 33.789 | 38.563 |
| 8 | 19.974 | | 27.563 | 34.032 | 37.720 |
| 9 | 19.894 | | 28.993 | 36.618 | 39.216 |
| 10 | 19.668 | | 27.632 | 33.558 | 36.974 |
| 11 | 19.839 | | 27.704 | 35.274 | 37.760 |
| 12 | 19.773 | | 29.139 | 36.295 | 41.285 |
| 13 | 19.618 | | 28.004 | 36.238 | 38.382 |
| 14 | 20.198 | | 26.732 | 34.345 | 37.328 |
| 15 | 19.948 | 22.853 | 27.523 | 33.143 | 37.100 |
| 16 | 19.882 | 23.175 | 27.851 | 33.421 | 37.084 |
| 17 | 19.975 | 21.639 | 28.195 | | 38.261 |
| 18 | 20.009 | 21.978 | 27.321 | | 38.568 |
| 19 | 20.087 | 21.892 | 28.309 | | 38.578 |
| 20 | 20.239 | 21.531 | 28.025 | | 38.253 |

35° C.

Table 6e
Total basic variants at 5° C.

| Formulation | T = 0 | 2M | 3M | 6M | 9M | 12M | 18M |
|---|---|---|---|---|---|---|---|
| 1 | 3.345 | 1.911 | 2.137 | 2.675 | | | |
| 2 | 3.084 | 1.740 | 2.187 | 2.573 | | | |
| 3 | 2.693 | 1.781 | 2.011 | 2.817 | | | |
| 4 | 3.132 | 1.745 | 1.856 | 2.600 | | | |
| 5 | 2.657 | 1.686 | 1.867 | 2.157 | | | |
| 6 | 3.113 | 1.647 | 1.971 | 2.416 | | | |
| 7 | 2.321 | 1.923 | 1.947 | 2.663 | | | |
| 8 | 2.679 | 1.765 | 2.036 | 2.652 | | | |
| 9 | 2.787 | 2.001 | 2.041 | 2.935 | | | |
| 10 | 2.681 | 1.880 | 2.121 | 3.376 | | | |
| 11 | 2.696 | 1.925 | 1.994 | 2.698 | | | |
| 12 | 2.735 | 1.621 | 2.126 | 2.799 | | | |
| 13 | 3.192 | 2.015 | 2.103 | 2.948 | | | |
| 14 | 3.444 | 1.678 | 1.922 | 2.544 | | | |
| 15 | 3.194 | | 1.942 | 2.692 | 3.107 | 3.132 | 2.929 |
| 16 | 2.638 | | 2.065 | 2.657 | 2.963 | 3.445 | |
| 17 | 3.535 | | 1.991 | 2.673 | 3.004 | | |
| 18 | 2.762 | | 1.902 | 2.688 | 2.961 | 2.927 | |
| 19 | 2.748 | | 1.868 | 2.481 | 2.843 | 3.052 | |
| 20 | 3.355 | | 2.004 | 2.805 | 2.876 | 3.077 | 2.781 |

TABLE 6f
Total basic variants at 15° C.

| Formulation | T = 0 | 2M | 3M |
|---|---|---|---|
| 1 | 3.3451 | 1.8877 | 2.1874 |
| 2 | 3.0845 | 1.8007 | 2.2426 |
| 3 | 2.6937 | 1.9116 | 2.3912 |
| 4 | 3.1325 | 1.7506 | 1.8685 |
| 5 | 2.6578 | 1.8572 | 1.9129 |
| 6 | 3.1131 | 1.7525 | 2.141 |
| 7 | 2.3214 | 2.1016 | 2.1524 |
| 8 | 2.6791 | 1.8722 | 2.3529 |
| 9 | 2.787 | 2.1082 | 2.4655 |
| 10 | 2.6818 | 1.9459 | 2.2071 |
| 11 | 2.6967 | 2.0282 | 2.4248 |
| 12 | 2.7351 | 1.7888 | 1.9481 |
| 13 | 3.1926 | 2.1603 | 2.2731 |
| 14 | 3.4443 | 1.9555 | 2.0979 |
| 15 | 3.1947 | | |
| 16 | 2.6381 | | |
| 17 | 3.5356 | | |
| 18 | 2.7621 | | |
| 19 | 2.7485 | | |
| 20 | 3.3555 | | |

15° C.

TABLE 6g
Total basic variants at 25° C.

| Formulation | T = 0 | 1M | 2M | 3M | 6M |
|---|---|---|---|---|---|
| 1 | 3.345 | 2.178 | 2.250 | 2.915 | 4.027 |
| 2 | 3.084 | 2.091 | 2.340 | 2.837 | 3.311 |
| 3 | 2.693 | 2.670 | 2.584 | 3.529 | 4.648 |
| 4 | 3.132 | 2.181 | 2.099 | 2.319 | 2.324 |
| 5 | 2.657 | 2.304 | 2.038 | 2.404 | 2.524 |
| 6 | 3.113 | 2.017 | 2.078 | 2.350 | 2.523 |
| 7 | 2.321 | 2.062 | 2.200 | 2.364 | 2.444 |
| 8 | 2.679 | 2.731 | 2.655 | 3.379 | 4.630 |
| 9 | 2.787 | 2.466 | 2.704 | 3.352 | 4.848 |
| 10 | 2.681 | 2.334 | 2.347 | 2.933 | 4.014 |
| 11 | 2.696 | 2.436 | 2.889 | 3.422 | 4.703 |
| 12 | 2.735 | 1.997 | 1.985 | 2.501 | 2.461 |
| 13 | 3.192 | 2.239 | 2.577 | 4.013 | 4.318 |
| 14 | 3.444 | 2.152 | 2.362 | 3.327 | 3.405 |
| 15 | 3.194 | 2.138 | | 3.224 | |
| 16 | 2.638 | 2.164 | | 3.158 | |
| 17 | 3.535 | 2.240 | | 3.230 | |
| 18 | 2.762 | 2.426 | | 3.203 | |
| 19 | 2.748 | 2.320 | | 3.138 | |
| 20 | 3.355 | 2.024 | | 3.137 | |

25° C.

TABLE 6g
Total basic variants at 35° C.

| Formulation | T = 0 | 0.5M | 1M | 2M | 3M |
|---|---|---|---|---|---|
| 1 | 3.345 | | 2.876 | 2.974 | 3.794 |
| 2 | 3.084 | | 2.946 | 2.919 | 3.902 |
| 3 | 2.693 | | 3.598 | 3.937 | 6.772 |
| 4 | 3.132 | | 2.416 | 2.212 | 2.204 |
| 5 | 2.657 | | 2.492 | 2.173 | 2.365 |
| 6 | 3.113 | | 2.276 | 2.201 | 2.474 |
| 7 | 2.321 | | 2.264 | 2.221 | 2.515 |
| 8 | 2.679 | | 3.180 | 3.936 | 6.582 |
| 9 | 2.787 | | 3.239 | 3.803 | 6.169 |
| 10 | 2.681 | | 2.659 | 3.093 | 3.647 |
| 11 | 2.696 | | 3.252 | 3.767 | 6.370 |
| 12 | 2.735 | | 2.246 | 2.058 | 2.300 |
| 13 | 3.192 | | 3.072 | 3.619 | 5.807 |
| 14 | 3.444 | | 2.864 | 2.789 | 4.317 |
| 15 | 3.194 | 2.726 | 2.809 | 2.968 | 4.349 |
| 16 | 2.638 | 2.720 | 2.691 | 3.046 | 4.312 |
| 17 | 3.535 | 2.827 | 2.780 | | 3.481 |
| 18 | 2.762 | 2.675 | 2.892 | | 3.544 |
| 19 | 2.748 | 2.609 | 3.099 | | 3.330 |
| 20 | 3.355 | 2.788 | 2.767 | | 3.579 |

35° C.

### Formulation Study A: Results—CE-SDS

The CE-SDS reduced percent purity values at 5° C., 15° C., 25° C. and 35° C. are shown in Tables 7a-7d.

An apparent increase in purity is observed for Formulations 15, 16, 19 and 20 from initial to three months at 5° C., which may be attributable to formulation to formulation variability. Those increases suggest that changes at 35° C. may be somewhat masked by the same systematic variability. Nonetheless, significant changes were not observed at 5° C. through 18 months and the overall changes after 3 months at 35° C. were <3%. With the high purity levels, both fragments and aggregates were low over the course of the study.

Projections of change in percent purity by reduced CE-SDS at 24-months with 5° C. storage have large uncertainty compared to the input variable trends. Protein concentration was the only statistically significant effect. All projections of purity across the study range at 24-months at 5° C. were <1% different from the initial value.

The CE-SDS non-reduced percent purity values at 5° C., 15° C., 25° C. and 35° C. are shown in Tables 7e-7h.

Similar to reduced CE-SDS, systematic variation appears to play a role in the results with apparent increases at 5° C. and 25° C. The increases suggest that changes at 35° C. may be somewhat masked by the same systematic variability. Nonetheless, significant changes were not observed at 5° C. through 18 months and the overall change after 3 months at 35° C. was <2%, similar to the reduced CE-SDS results. Aggregates did not show any trend over the course of the study; however, fragments increased at 35° C. commensurate with decreasing purity. Among the input variables affecting percent purity by non-reduced CE-SDS, only antibody concentration and container closure were significant. The highest predicted purity is at pH 5.5. In all cases the effects of the input variables was <1.2% differences for the 24-month predictions.

TABLE 7a
CE-SDS Reduced Percent Purity at 5° C.

| Formu- | | | | | | | |
|---|---|---|---|---|---|---|---|
| lation | T = 0 | 2M | 3M | 6M | 9M | 12M | 18M |
| 1 | 97.550 | 98.380 | 98.281 | 97.810 | | | |
| 2 | 97.064 | 97.973 | 98.154 | 97.360 | | | |
| 3 | 97.452 | 97.323 | 98.490 | 98.114 | | | |
| 4 | 97.827 | 97.776 | 97.688 | 97.843 | | | |
| 5 | 97.394 | 98.158 | 98.105 | 98.221 | | | |
| 6 | 97.657 | 97.976 | 98.232 | 97.858 | | | |
| 7 | 97.758 | 97.120 | 98.006 | 97.880 | | | |
| 8 | 97.481 | 98.029 | 98.153 | 98.186 | | | |
| 9 | 97.540 | 98.030 | 98.363 | 98.140 | | | |
| 10 | 97.106 | 97.435 | 97.096 | 97.334 | | | |
| 11 | 97.654 | 97.322 | 97.672 | 97.714 | | | |
| 12 | 97.161 | 97.948 | 97.801 | 98.277 | | | |
| 13 | 96.832 | 97.594 | 97.561 | 98.026 | | | |
| 14 | 97.109 | 98.062 | 98.248 | 97.720 | | | |
| 15 | 96.584 | | 98.303 | 97.969 | 97.839 | 98.496 | 98.478 |
| 16 | 96.623 | | 98.205 | 98.313 | 97.833 | 98.494 | |
| 17 | 96.734 | | 98.013 | 97.847 | 97.833 | | |
| 18 | 97.229 | | 97.931 | 98.260 | 97.841 | 98.727 | |
| 19 | 97.457 | | 97.561 | 98.172 | 98.313 | 98.612 | |
| 20 | 96.865 | | 98.211 | 98.395 | 98.290 | 98.361 | 98.337 |

TABLE 7b
CE-SDS Reduced Percent Purity at 15° C.

| Formulation | T = 0 | 2M | 3M |
|---|---|---|---|
| 1 | 97.550 | 98.382 | 98.128 |
| 2 | 97.064 | 98.143 | 97.964 |
| 3 | 97.452 | 97.487 | 97.402 |
| 4 | 97.827 | 96.961 | 97.284 |
| 5 | 97.394 | 97.505 | 97.549 |
| 6 | 97.657 | 98.169 | 97.256 |
| 7 | 97.758 | 97.269 | 97.368 |
| 8 | 97.481 | 97.350 | 97.338 |
| 9 | 97.540 | 98.188 | 97.713 |
| 10 | 97.106 | 97.343 | 97.457 |
| 11 | 97.654 | 97.396 | 97.346 |
| 12 | 97.161 | 97.899 | 96.894 |
| 13 | 96.832 | 97.163 | 97.951 |
| 14 | 97.109 | 97.936 | 97.955 |
| 15 | 96.584 | | |
| 16 | 96.623 | | |
| 17 | 96.734 | | |
| 18 | 97.229 | | |
| 19 | 97.457 | | |
| 20 | 96.865 | | |

TABLE 7c
CE-SDS Reduced Percent Purity at 25° C.

| Formulation | T = 0 | 1M | 2M | 3M | 6M |
|---|---|---|---|---|---|
| 1 | 97.550 | 97.718 | 97.898 | 97.378 | 97.002 |
| 2 | 97.064 | 97.529 | 97.706 | 97.398 | 96.757 |
| 3 | 97.452 | 97.003 | 97.202 | 97.652 | 96.057 |
| 4 | 97.827 | 97.360 | 96.005 | 97.265 | 96.694 |
| 5 | 97.394 | 96.521 | 97.354 | 97.152 | 96.305 |
| 6 | 97.657 | 97.223 | 97.371 | 96.671 | 97.133 |
| 7 | 97.758 | 97.498 | 96.866 | 96.982 | 96.820 |
| 8 | 97.481 | 96.903 | 97.005 | 97.050 | 96.431 |
| 9 | 97.540 | 97.592 | 97.964 | 97.285 | 97.031 |
| 10 | 97.106 | 97.135 | 97.420 | 97.202 | 97.023 |
| 11 | 97.654 | 97.429 | 96.908 | 97.631 | 96.693 |
| 12 | 97.161 | 97.162 | 97.770 | 97.190 | 96.763 |
| 13 | 96.832 | 97.239 | 96.997 | 97.032 | 96.095 |
| 14 | 97.109 | 97.383 | 97.822 | 97.467 | 97.086 |
| 15 | 96.584 | 97.314 | | 97.654 | |
| 16 | 96.623 | 97.190 | | 97.655 | |
| 17 | 96.734 | 97.559 | | 96.936 | |
| 18 | 97.229 | 97.117 | | 96.494 | |
| 19 | 97.457 | 97.037 | | 96.185 | |
| 20 | 96.865 | 96.990 | | 97.551 | |

25° C.

TABLE 7d
CE-SDS Reduced Percent Purity at 35° C.

| Formulation | T = 0 | 0.5M | 1M | 2M | 3M |
|---|---|---|---|---|---|
| 1 | 97.550 | 97.550 | 96.931 | 97.287 | 96.153 |
| 2 | 97.064 | | 97.475 | 97.169 | 96.068 |
| 3 | 97.452 | | 97.247 | 96.730 | 94.431 |
| 4 | 97.827 | | 96.703 | 95.666 | 95.929 |
| 5 | 97.394 | | 96.968 | 96.982 | 95.005 |
| 6 | 97.657 | | 97.129 | 97.125 | 96.174 |
| 7 | 97.758 | | 96.973 | 95.771 | 95.682 |
| 8 | 97.481 | | 96.567 | 96.391 | 95.462 |
| 9 | 97.540 | | 96.737 | 96.917 | 95.451 |
| 10 | 97.106 | | 96.737 | 95.906 | 95.688 |
| 11 | 97.654 | | 96.803 | 95.624 | 94.906 |
| 12 | 97.161 | | 96.865 | 96.792 | 95.993 |
| 13 | 96.832 | | 95.928 | 95.582 | 95.078 |
| 14 | 97.109 | | 96.866 | 97.074 | 96.075 |
| 15 | 96.584 | 97.706 | 97.454 | 96.900 | 95.929 |
| 16 | 96.623 | 97.452 | 97.658 | 96.239 | 96.033 |
| 17 | 96.734 | 97.506 | 97.378 | | 95.694 |
| 18 | 97.229 | 97.176 | 96.873 | | 95.819 |
| 19 | 97.457 | 97.032 | 96.548 | | 96.147 |
| 20 | 96.865 | 97.659 | 96.371 | | 95.613 |

35° C.

TABLE 7e
CE-SDS Non-Reduced Percent Purity at 5° C.

| Formu- | | | | | | | |
|---|---|---|---|---|---|---|---|
| lation | T = 0 | 2M | 3M | 6M | 9M | 12M | 18M |
| 1 | 96.903 | 96.844 | 98.362 | 98.204 | | | |
| 2 | 97.297 | 97.458 | 98.562 | 98.382 | | | |
| 3 | 97.344 | 96.963 | 98.432 | 98.283 | | | |
| 4 | 96.591 | 96.683 | 98.078 | 98.048 | | | |
| 5 | 97.070 | 97.072 | 98.400 | 98.423 | | | |
| 6 | 96.604 | 96.724 | 98.168 | 98.019 | | | |
| 7 | 96.999 | 96.527 | 97.426 | 97.635 | | | |
| 8 | 97.359 | 97.109 | 98.376 | 98.297 | | | |
| 9 | 97.033 | 96.929 | 98.221 | 98.239 | | | |
| 10 | 96.941 | 96.805 | 98.031 | 97.874 | | | |
| 11 | 97.409 | 97.048 | 97.866 | 98.052 | | | |
| 12 | 97.049 | 96.927 | 98.243 | 98.128 | | | |
| 13 | 96.994 | 96.676 | 98.162 | 98.021 | | | |
| 14 | 97.383 | 97.161 | 98.161 | 98.167 | | | |
| 15 | 96.736 | | 98.204 | 98.009 | 97.969 | 98.321 | 98.203 |
| 16 | 96.773 | | 97.918 | 98.094 | 97.769 | 98.121 | |
| 17 | 96.929 | | 98.071 | 98.223 | 97.928 | | |
| 18 | 96.499 | | 98.299 | 98.236 | 98.058 | 98.148 | |
| 19 | 96.609 | | 98.454 | 98.227 | 98.007 | 98.152 | |
| 20 | 96.735 | | 97.886 | 97.788 | 97.634 | 98.396 | 97.690 |

TABLE 7f
CE-SDS Non-Reduced Percent Purity at 15° C.

| Formulation | T = 0 | 2M | 3M |
|---|---|---|---|
| 1 | 96.903 | 96.971 | 97.965 |
| 2 | 97.297 | 96.904 | 98.291 |
| 3 | 97.344 | 96.913 | 98.189 |
| 4 | 96.591 | 96.623 | 97.688 |
| 5 | 97.070 | 96.602 | 98.430 |
| 6 | 96.604 | 96.438 | 98.183 |
| 7 | 96.999 | 96.330 | 97.922 |
| 8 | 97.359 | 97.206 | 98.481 |
| 9 | 97.033 | 96.965 | 98.042 |
| 10 | 96.941 | 96.915 | 98.168 |
| 11 | 97.409 | 97.049 | 97.884 |
| 12 | 97.049 | 96.834 | 98.587 |
| 13 | 96.994 | 96.806 | 98.205 |
| 14 | 97.383 | 97.152 | 98.378 |
| 15 | 96.736 | | |
| 16 | 96.773 | | |
| 17 | 96.929 | | |
| 18 | 96.499 | | |
| 19 | 96.609 | | |
| 20 | 96.735 | | |

TABLE 7g
CE-SDS Non-Reduced Percent Purity at 25° C.

| Formulation | T = 0 | 1M | 2M | 3M | 6M |
|---|---|---|---|---|---|
| 1 | 96.903 | 96.247 | 96.473 | 97.569 | 95.584 |
| 2 | 97.297 | 96.460 | 96.882 | 97.830 | 96.279 |
| 3 | 97.344 | 96.117 | 96.870 | 97.720 | 96.067 |
| 4 | 96.591 | 95.357 | 96.769 | 97.385 | 96.053 |
| 5 | 97.070 | 96.299 | 96.473 | 97.440 | 96.484 |
| 6 | 96.604 | 95.749 | 96.482 | 97.250 | 96.157 |
| 7 | 96.999 | 95.740 | 95.955 | 97.108 | 95.577 |
| 8 | 97.359 | 96.330 | 95.014 | 97.484 | 95.854 |
| 9 | 97.033 | 96.093 | 96.399 | 97.392 | 95.530 |
| 10 | 96.941 | 95.732 | 96.189 | 97.210 | 95.793 |
| 11 | 97.409 | 95.630 | 95.645 | 96.955 | 95.077 |
| 12 | 97.049 | 96.215 | 95.912 | 97.574 | 96.321 |
| 13 | 96.994 | 95.577 | 95.923 | 97.090 | 95.220 |
| 14 | 97.383 | 95.767 | 95.914 | 97.608 | 95.831 |
| 15 | 96.736 | 95.757 | | 97.322 | |
| 16 | 96.773 | 95.489 | | 97.235 | |
| 17 | 96.929 | 95.731 | | 97.541 | |
| 18 | 96.499 | 96.073 | | 97.753 | |
| 19 | 96.609 | 96.017 | | 97.740 | |
| 20 | 96.735 | 95.975 | | 97.643 | |

25° C.

TABLE 7h
CE-SDS Non-Reduced Percent Purity at 35° C.

| Formulation | T = 0 | 0.5M | 1M | 2M | 3M |
|---|---|---|---|---|---|
| 1 | 96.903 | | 95.454 | 95.721 | 95.995 |
| 2 | 97.297 | | 96.630 | 96.486 | 96.484 |
| 3 | 97.344 | | 96.141 | 94.058 | 95.166 |
| 4 | 96.591 | | 95.567 | 95.263 | 95.732 |
| 5 | 97.070 | | 95.816 | 95.163 | 95.843 |
| 6 | 96.604 | | 95.766 | 95.579 | 95.938 |
| 7 | 96.999 | | 95.382 | 94.975 | 95.089 |
| 8 | 97.359 | | 96.053 | 94.639 | 95.015 |
| 9 | 97.033 | | 95.943 | 94.924 | 94.719 |
| 10 | 96.941 | | 96.058 | 95.089 | 95.533 |
| 11 | 97.409 | | 95.868 | 94.826 | 94.168 |
| 12 | 97.049 | | 95.963 | 95.909 | 96.374 |
| 13 | 96.994 | | 95.614 | 94.861 | 94.963 |
| 14 | 97.383 | | 95.630 | 95.484 | 96.097 |
| 15 | 96.736 | 96.762 | 95.920 | 95.068 | 95.814 |
| 16 | 96.773 | 96.554 | 95.633 | 94.996 | 95.630 |
| 17 | 96.929 | 96.688 | 95.518 | | 95.580 |
| 18 | 96.499 | 95.063 | 95.758 | | 95.868 |
| 19 | 96.609 | 95.862 | 95.632 | | 95.657 |
| 20 | 96.735 | 96.623 | 95.785 | | 95.223 |

35° C.

### Formulation Study A: Results—Subvisible Particles

### a) HIAC

The data from HIAC subvisible particle testing at 5° C., 15° C., 25° C. and 35° C. is shown in Tables 8a-8d.

Most formulations at 25° C. through 3 months have counts below 5000, which is within the acceptable range for subvisible counts in a prefilled syringe. Formulation Nos. 4, 7, 10, 11 and 13 have values that are well in excess of this count. These formulations are the five formulations that have an antibody target concentration of 150 mg/mL. The next closest formulation in terms of less than 2 μm/mL counts is Formulation No. 16, which has an antibody concentration of 125 mg/mL. Formulation No. 4 has the greatest number of particles and the highest values are not fully reliable as they exceed the qualified range of the instrument. Subvisible counts at an antibody concentration of 150 mg/mL are also higher than other runs at 5° C. but the trend is more pronounced at 25° C. Notably, Formulation Nos. 4, 7, 10, 11 and 13 still conform to USP <788>count/container requirements throughout the study apart from the 3-month 35° C. time point.

TABLE 8a
HIAC less than or equal to 2 μm counts/mL at 5° C.

| Formulation No. | T = 0 | 3M | 12 | 18M |
|---|---|---|---|---|
| 1 | 206 | 162 | | |
| 2 | 123 | 330 | | |
| 3 | 128 | 203 | | |
| 4 | 1543 | 1556 | | |
| 5 | 128 | 208 | | |
| 6 | 112 | 289 | | |
| 7 | 1034 | 1510 | | |
| 8 | 295 | 163 | | |
| 9 | 165 | 959 | | |
| 10 | 764 | 746 | | |
| 11 | 854 | 1114 | | |
| 12 | 190 | 256 | | |
| 13 | 996 | 416 | | |
| 14 | 240 | 146 | | |
| 15 | 147 | 109 | 1890 | 4269 |
| 16 | 339 | 181 | 951 | |
| 17 | 199 | 284 | | |
| 18 | 182 | 826 | 3002 | |
| 19 | 215 | 683 | | |
| 20 | 354 | 305 | 2779 | 8107 |

5° C.

TABLE 8b
HIAC less than or equal to 2 μm counts/mL at 15° C.

| Formulation | T = 0 | 3M |
|---|---|---|
| 1 | 206 | 984 |
| 2 | 123 | 2362 |
| 3 | 128 | 1358 |
| 4 | 154 | 20270 |
| 5 | 128 | 1388 |
| 6 | 112 | 5494 |
| 7 | 103 | 8260 |
| 8 | 295 | 2078 |
| 9 | 165 | 1053 |
| 10 | 764 | 11392 |
| 11 | 854 | 7273 |
| 12 | 190 | 2259 |
| 13 | 996 | 9403 |
| 14 | 240 | 1522 |
| 15 | 147 | |
| 16 | 339 | |
| 17 | 199 | |
| 18 | 182 | |
| 19 | 215 | |
| 20 | 354 | |

5° C.

TABLE 8c
HIAC less than or equal to 2 μm counts/mL at 25° C.

| Formulation No. | T = 0 | 1M | 3M |
|---|---|---|---|
| 1 | 2066 | 1448 | 1294 |
| 2 | 1237 | 3486 | 1708 |
| 3 | 1289 | 1834 | 1896 |
| 4 | 1543 | 32874 | 39721 |
| 5 | 1285 | 1856 | 1547 |
| 6 | 1124 | 4729 | 2015 |
| 7 | 1034 | 12181 | 7313 |
| 8 | 2958 | 1784 | 3614 |
| 9 | 1655 | 1371 | 3001 |
| 10 | 7644 | 6200 | 3307 |
| 11 | 8549 | 4977 | 11507 |
| 12 | 1903 | 5037 | 3280 |
| 13 | 9961 | 5323 | 16237 |
| 14 | 2402 | 2750 | 2461 |
| 15 | 1470 | 2919 | 1422 |
| 16 | 3394 | 3363 | 5602 |
| 17 | 199 | 332 | 403 |
| 18 | 182 | 335 | 227 |
| 19 | 215 | 490 | 1256 |
| 20 | 3545 | 3105 | 3743 |

25° C.

TABLE 8d
HIAC less than or equal to 2 μm counts/mL at 25° C.

| Formulation No. | T = 0 | 1M | 3M |
|---|---|---|---|
| 1 | 2066 | 1157 | 2880 |
| 2 | 1237 | 1317 | 2582 |
| 3 | 1289 | 2258 | 4423 |
| 4 | 1543 | 23054 | 43613 |
| 5 | 1285 | 2070 | 1992 |
| 6 | 1124 | 2604 | 4533 |
| 7 | 1034 | 8965 | 18476 |
| 8 | 2958 | 2911 | 8252 |
| 9 | 1655 | 1980 | 5162 |
| 10 | 7644 | 20558 | 34576 |
| 11 | 8549 | 9866 | 29565 |
| 12 | 1903 | 3500 | 3693 |
| 13 | 9961 | 20738 | 28594 |
| 14 | 2402 | 5691 | 1949 |
| 15 | 1470 | 2889 | 3740 |
| 16 | 3394 | 2492 | 4543 |
| 17 | 199 | 507 | 1581 |
| 18 | 182 | 514 | 1153 |
| 19 | 215 | 685 | 2205 |
| 20 | 3545 | 4855 | 8602 |

35° C.

### b) MFI

The data from MFI subvisible particle testing at 5° C., 15° C., 25° C. and 35° C. is shown in Tables 8e-8g.

Similar trends were observed with MFI results as compared to HIAC results. At 25° C., the highest counts across all of the formulations correspond to those with an antibody concentration of 150 mg/mL. Unlike HIAC results, Formulation No. 16 counts were comparable to those of lower antibody concentration formulations. Formulation No. 4 again showed the highest counts (nearly an order of magnitude higher than other formulations).

TABLE 8e
MFI less than or equal to 2 μm counts/mL at 5° C.

| Formulation | T = 0 | 3M | 6M | 18M |
|---|---|---|---|---|
| 1 | 1316 | 4437 | | |
| 2 | 5718 | 31942 | 9000 | |
| 3 | 8812 | 6348 | 11010 | |
| 4 | 151896 | 159322 | | |
| 5 | 6748 | 4689 | 3116 | |
| 6 | 7006 | 16263 | | |
| 7 | 80650 | 48541 | | |
| 8 | 17001 | 5683 | 2526 | |
| 9 | 933 | 2386 | | |
| 10 | 24888 | 26509 | | |
| 11 | 29499 | 40247 | | |
| 12 | 27221 | 11188 | | |
| 13 | 38049 | 8242 | | |
| 14 | 5480 | 2218 | | |
| 15 | 1038 | 2035 | | 64155 |
| 16 | 3158 | | | |
| 17 | 9250 | 1332 | | |
| 18 | 5417 | 5500 | | |
| 19 | 2737 | 2584 | | |
| 20 | 1796 | 5164 | | 3940 |

5° C.

TABLE 8f
MFI less than or equal to 2 μm counts/mL at 25° C.

| Formulation | T = 0 | 3M | 6M |
|---|---|---|---|
| 1 | 1316 | 2028 | 8496 |
| 2 | 5718 | 4630 | 11086 |
| 3 | 8812 | 12999 | 9110 |
| 4 | 15189 | 45468 | 80501 |
| 5 | 6748 | 9283 | 32884 |
| 6 | 7006 | 11535 | 10671 |
| 7 | 80650 | 30786 | 73491 |
| 8 | 17001 | 11595 | 17139 |
| 9 | 933 | 7622 | 9875 |
| 10 | 24888 | 8900 | 23889 |
| 11 | 29499 | 23468 | |
| 12 | 27221 | 16450 | 20885 |
| 13 | 38049 | 72204 | 41426 |
| 14 | 5480 | 4936 | 13277 |
| 15 | 1038 | 4995 | |
| 16 | 3158 | 5791 | |
| 17 | 9250 | 3649 | |
| 18 | 5417 | 4114 | |
| 19 | 2737 | 2866 | |
| 20 | 1796 | | |

25° C.

TABLE 8g
MFI less than or equal to 2 μm counts/mL at 35° C.

| Formulation | T = 0 | 3M |
|---|---|---|
| 1 | 1316 | 14816 |
| 2 | 5718 | 15479 |
| 3 | 8812 | |
| 4 | 151896 | 447247 |
| 5 | 6748 | 5677 |
| 6 | 7006 | 68495 |
| 7 | 80650 | 116643 |
| 8 | 17001 | 26143 |
| 9 | 933 | 19053 |
| 10 | 24888 | 371523 |
| 11 | 29499 | 98003 |
| 12 | 27221 | 12521 |
| 13 | 38049 | 119192 |
| 14 | 5480 | 5572 |
| 15 | 1038 | 12973 |
| 16 | 3158 | 12246 |
| 17 | 9250 | |
| 18 | 5417 | |
| 19 | 2737 | |
| 20 | 1796 | |

35° C.

### 5 Formulation Study A: Conclusions

The purpose of Formulation Study A was to identify a formulation composition suitable for administration to human patients and to monitor the robustness of the formulation by systematically optimizing the critical formulation parameters with respect to stability properties. In this study, physical and chemical stability were evaluated as functions of mirikizumab concentration, pH, NaCl and polysorbate 80. Several formulations appear to be robust from a chemical and physical stability standpoint over the entire region studied with all 24-month at 5° C. change projections <5%. Optimal stability by SEC is closer to pH 5.0 (though the entire pH range had changes <2% after 24-months at 5° C.). iCIEF results indicated that optimal stability was between pH 5.5 and pH 6.0. Other methods did not show clear trends for pH. Accounting for these projections, pH 5.5 is deemed to be the optimal pH since it balances the observations from both relevant assays. Increasing protein concentration did result in lower SEC percent monomer and lower non-reduced CE-SDS purity but the differences between 20 and 150 mg/mL were <1%. No significant trends were observed in relation to changes in NaCl or polysorbate 80 concentrations. There were also no significant effects observed between container closure types in this study. Subvisible particle counts were higher in the formulations targeting 150 mg/mL of mirikizumab. Additional studies are being undertaken to better understand the causes of this observation. Based on results described here, the preferred formulation is 10 mM citrate buffer, 150 mM NaCl, 0.03% w/v polysorbate 80 (0.05% w/v in vials for IV administration) at pH 5.5. For intravenous administration from vials, the preferred concentration of polysorbate 80 is 0.05% w/v.

### Example 3: Formulation Study B

### Purpose

It has been hypothesized that the presence of sodium chloride and/or citrate may increase the likelihood of injection site discomfort. The purpose of Formulation Study B is to identify an alternative formulation of mirikizumab that has a high probability of providing a well-tolerated injection experience. In addition to improving perceived injection pain, other objectives of the study include: meeting standard bioequivalence criteria compared to the preferred formulation identified in Formulation Study A and maintaining and/or minimally perturbing the stability, manufacturability, and deliverability afforded by the preferred formulation.

### Formulation Study B: Study Design and Preparation of Anti-IL-23p19 Antibody Pharmaceutical Formulations

Part I of the study comprised the design and assessment of a number of formulations as shown in Table 9a.

TABLE 9a
Formulations assessed in Part A of Formulation Study B

| Formula- | Antibody | | | | |
|---|---|---|---|---|---|
| tion No. | (mg/mL) | Buffer | Excipients | Surfactant | pH¹ |
| 1 | 125 | 10 mM | 150 mM NaCl | 0.03% w/v | 5.5 |
| | | citrate | | PS80 | |
| 21 | 125 | 5 mM | 5% w/v | 0.03% w/v | 5.5 |
| | | citrate | mannitol | PS80 | |
| 22 | 125 | 5 mM | 5% w/v | 0.03% w/v | 5.6 |
| | | histidine | mannitol | PS80 | |
| 23 | 125 | 5 mM | 5% w/v | 0.03% w/v | 5.9 |
| | | histidine | mannitol | PS80 | |
| 24 | 125 | 5 mM | 5% w/v | 0.03% w/v | 6.2 |
| | | histidine | mannitol | PS80 | |
| 25 | 125 | 10 mM | 37.5 mM NaCl | 0.03% w/v | 5.5 |
| | | histidine | 4.1% w/v | PS80 | |
| | | | mannitol | | |
| 26 | 125 | 10 mM | 75 mM NaCl | 0.03% w/v | 5.5 |
| | | histidine | 3.3% w/v | PS80 | |
| | | | mannitol | | |
| 27 | 125 | 5 mM | 9% w/v | 0.03% w/v | 5.6 |
| | | histidine | sucrose | PS80 | |
| 28 | 125 | 5 mM | 9% w/v | 0.03% w/v | 5.6 |
| | | histidine | trehalose | PS80 | |
| 29 | 125 | self- | 5% w/v | 0.03% w/v | 5.4 |
| | | buffered | mannitol | PS80 | |

¹Average of measured values across all stability conditions

With the exception of Formulation 1 (which is the preferred formulation from Formulation Study A), samples were prepared by buffer exchange of drug substance lot

EL01685-056-F-Fill (C1 demo #2) into the matrices (without polysorbate 80) listed in Table 9. The buffer exchanged samples were concentrated and/or diluted with buffer to 125 mg/mL of mirikizumab, and spiked with polysorbate 80 (PS80) to a final concentration of 0.03% w/v. The formulations were then sterile filtered, filled into a 2.25 mL syringe, and the appropriate plunger inserted. The final drug product samples were stored and pulled from chambers as indicated in Table 9b.

TABLE 9b
Part A Time Point and Temperature Conditions

| | Time (weeks) | | | | | |
|---|---|---|---|---|---|---|
| Temperature (° C.) | 0 | 2 | 4 | 8 | 13 | 26 |
| 5 | X | | X | | X | X |
| 25 | | | X | X | X | |
| 40 | | X | X | X | | |

The results from the assessment of the formulations shown in Table 9a led to design and assessment of further formulations as shown in Table 10a (Part II of the study).

TABLE 10a
Formulations assessed in Part B of Formulation Study B

| Formula- | Antibody | | | | |
|---|---|---|---|---|---|
| tion No. | (mg/mL) | Buffer | Excipients | Surfactant | pH¹ |
| 1 | 125 | 10 mM | 150 mM NaCl | 0.03% w/v | 5.5 |
| | | citrate | | PS80 | |
| 30 | 125 | 5 mM | 25 mM NaCl | 0.03% w/v | 5.9 |
| | | histidine | 4.1% w/v | PS80 | |
| | | | mannitol | | |
| 31 | 125 | self- | 25 mM NaCl | 0.03% w/v | 5.3 |
| | | buffered | 4.1% w/v | PS80 | |
| | | | mannitol | | |
| 32 | 125 | 5 mM | 25 mM NaCl | 0.03% w/v | 5.2 |
| | | histidine | 4.1% w/v | PS80 | |
| | | | mannitol | | |
| 33 | 125 | 5 mM | 25 mM NaCl | 0.03% w/v | 6.3 |
| | | histidine | 4.1% w/v | PS80 | |
| | | | mannitol | | |
| 34 | 125 | 5 mM | 25 mM NaCl | 0.03% w/v | 5.6 |
| | | histidine | 4.1% w/v | PS80 | |
| | | | mannitol | | |
| 35 | 125 | self- | 150 mM NaCl | 0.03% w/v | 5.5 |
| | | buffered | | PS80 | |
| 36 | 125 | self- | 25 mM NaCl | 0.03% w/v | 6.0 |
| | | buffered | 4.1% w/v | PS80 | |
| | | | mannitol | | |

¹Average of measured values across all stability conditions

With the exception of Formulation 1 (which is the preferred formulation from Formulation Study A), samples were prepared by buffer exchange (first against 0.3 M NaCl) of drug substance lot EL01685-056-F-Fill (C1 demo #2) against 0.3 M NaCl and then buffer exchanged further into the matrices (without polysorbate 80) listed in Table 10a. This two-step dialysis approach was used to limit the amount of residual citrate in the final drug product samples. The buffer exchanged samples were concentrated and/or diluted with buffer to 125 mg/mL of mirikizumab, and spiked with a PS80 to a final concentration of 0.03% w/v. The formulations were then sterile filtered, filled into a 2.25 mL syringe, and the appropriate plunger inserted. The final drug product samples were stored and pulled from chambers as indicated in Table 10b.

TABLE 10b
Part B Time Point and Temperature Conditions

| | Time (weeks) | | | | | |
|---|---|---|---|---|---|---|
| Temperature (° C.) | 0 | 2 | 4¹ | 8 | 13 | 26 |
| 5³ | X | | | X | X | X |
| 25 | | | X | X | X² | |
| 35 | | X | X | X | | |

¹Formulation 34 was submitted at week 5.
²Formulation 35 and Formulation 36 were submitted at week 14.
³Formulation 35 and Formulation 36 were submitted only at weeks 0 and 4 (the 4 week data will not be presented for these formulations).

The results from the assessment of the formulations shown in Table 9a and Table 10a led to design and assessment of further formulations as shown in Table 11a (Part III of the study).

TABLE 11a
Formulations assessed in Part C of Formulation Study B

| Formu- | | | | | | Drug |
|---|---|---|---|---|---|---|
| lation | Antibody | | | | | Substance |
| No. | (mg/mL) | Buffer | Excipients | Surfactant | pH¹ | Lot |
| 1 | 125 | 10 mM | 150 mM | 0.03% w/v | 5.5 | EL01685- |
| | | citrate | NaCl | PS80 | | 056-F-Fill |
| 37 | 125 | self- | 75 mM | 0.03% w/v | 5.5 | EL19481- |
| | | buffered | NaCl, | PS80 | | 008-F-Fill |
| | | | 2.5% w/v | | | |
| | | | mannitol | | | |
| 38 | 125 | self- | 75 mM | 0.03% w/v | 5.4 | EL01685- |
| | | buffered | NaCl, | PS80 | | 056-F-Fill |
| | | | 2.5% w/v | | | |
| | | | mannitol | | | |
| 39 | 125 | 5 mM | 30 mM | 0.03% w/v | 5.3 | EL19481- |
| | | histidine | NaCl, | PS80 | | 007-F-Fill |
| | | | 3.9% w/v | | | |
| | | | mannitol | | | |
| 40 | 125 | 5 mM | 50 mM | 0.03% w/v | 5.4 | EL19481- |
| | | histidine | NaCl, | PS80 | | 007-F-Fill |
| | | | 3.3% w/v | | | |
| | | | mannitol | | | |

¹Average of measured values across all stability conditions

With the exception of Formulation 1 (which is the preferred formulation from Formulation Study A), samples were prepared by buffer exchange or dilution of drug substance into the matrices (without polysorbate 80) listed in Table 11a. Formulation 38 was first dialyzed against 0.3 M NaCl. The samples were concentrated and/or diluted with buffer to 125 mg/mL of mirikizumab, and spiked with a PS80 to a final concentration of 0.03% w/v. The formulations were then sterile filtered, filled into the 2.25 mL syringe, and the appropriate plunger inserted. The final drug product samples were stored and pulled from chambers as indicated in Table 11b.

TABLE 11b
Part C Time Point and Temperature Conditions

| | Time (weeks) | | | | | |
|---|---|---|---|---|---|---|
| Temperature (° C.) | 0 | 2 | 4 | 8 | 12 | 26 |
| 5 | X | | X | | X | X |
| 25 | | | X | X | X | |
| 35 | | X¹ | X | X | | |

¹Formulation 39 was not submitted at week 2.

### Formulation Study B: Part I Results—Purity

Both SEC and both CE-SDS methods showed a time- and temperature-dependent decrease in mirikizumab purity. All test formulations performed comparably to or better than the Formulation 1. The non-histidine containing matrices (Formulations 1, 21 and 29) displayed the largest decreases in purity over the course of the stability study. The SEC monomer purity degradation rates at 25° C. and 40° C. are shown in Table 12. The non-histidine containing matrices (Formulations 1, 21 and 29) displayed the fastest degradation rates at the 25° C. and 40° C. conditions. Formulations 23 and 24 did not maintain solubility under refrigerated conditions.

TABLE 12
SEC Monomer Purity Degradation Rates at Elevated Temperature
(The data was fit to a simple linear regression to determine a degradation rate)

| | | | | Degradation | R | Degradation | R |
|---|---|---|---|---|---|---|---|
| Formulation | | | | Rate, 40° C. | squared, | Rate, 25° C. | squared, |
| No. | Buffer | Excipients | pH | (%/week) | 40° C. | (%/week) | 25° C. |
| 1 | 10 mM | 150 mM | 5.5 | −0.3157 | 0.9980 | −0.0370 | 0.9754 |
| | citrate | NaCl | | | | | |
| 21 | 5 mM | 5% w/v | 5.5 | −0.2774 | 0.9952 | −0.0286 | 0.9671 |
| | citrate | mannitol | | | | | |
| 22 | 5 mM | 5% w/v | 5.6 | −0.2005 | 0.9980 | −0.0219 | 0.9478 |
| | histidine | mannitol | | | | | |
| 23 | 5 mM | 5% w/v | 5.9 | −0.1887 | 0.9991 | −0.0261 | 0.9212 |
| | histidine | mannitol | | | | | |
| 24 | 5 mM | 5% w/v | 6.2 | −0.1911 | 0.9993 | −0.0275 | 0.8678 |
| | histidine | mannitol | | | | | |
| 25 | 10 mM | 37.5 mM | 5.5 | −0.2299 | 0.9969 | −0.0185 | 0.9527 |
| | histidine | NaCl 4.1% | | | | | |
| | | w/v | | | | | |
| | | mannitol | | | | | |
| 26 | 10 mM | 75 mM | 5.5 | −0.2517 | 0.9900 | −0.0231 | 0.9933 |
| | histidine | NaCl 3.3% | | | | | |
| | | w/v | | | | | |
| | | mannitol | | | | | |
| 27 | 5 mM | 9% w/v | 5.6 | −0.2160 | 0.9933 | −0.0197 | 0.8909 |
| | histidine | sucrose | | | | | |
| 28 | 5 mM | 9% w/v | 5.6 | −0.2124 | 0.9977 | −0.0201 | 0.9550 |
| | histidine | trehalose | | | | | |
| 29 | self- | 5% w/v | 5.4 | −0.3056 | 0.9934 | −0.0390 | 0.9120 |
| | buffered | mannitol | | | | | |

### Formulation Study B: Part I Results—Aggregates

SEC data showed a time- and temperature-dependent increase in mirikizumab aggregates. All formulations performed comparably to or better than Formulation 1. The non-histidine containing matrices (Formulations 1, 21, and 29) displayed the largest increases in aggregate over the course of the stability study. The SEC aggregates formation rates at 25° C. and 40° C. are shown in Table 13. The non-histidine containing matrices (Formulations 1, 21, and 29) displayed the fastest degradation rates at the 25° C. and 40° C. conditions.

TABLE 13
SEC Aggregates Formation Rates at Elevated Temperature

| | | | | Degradation | R | Degradation | R |
|---|---|---|---|---|---|---|---|
| Formulation | | | | Rate, 40° C. | squared, | Rate, 25° C. | squared, |
| No. | Buffer | Excipients | pH | (%/week) | 40° C. | (%/week) | 25° C. |
| 1 | 10 mM | 150 mM | 5.5 | 0.2464 | 0.9864 | 0.0322 | 0.9901 |
| | citrate | NaCl | | | | | |
| 21 | 5 mM | 5% w/v | 5.5 | 0.2249 | 0.9936 | 0.0255 | 0.9980 |
| | citrate | mannitol | | | | | |
| 22 | 5 mM | 5% w/v | 5.6 | 0.1654 | 0.9907 | 0.0191 | 0.9673 |
| | histidine | mannitol | | | | | |
| 23 | 5 mM | 5% w/v | 5.9 | 0.1609 | 0.9968 | 0.0232 | 0.9631 |
| | histidine | mannitol | | | | | |
| 24 | 5 mM | 5% w/v | 6.2 | 0.1683 | 0.9991 | 0.0238 | 0.9178 |
| | histidine | mannitol | | | | | |
| 25 | 10 mM | 37.5 mM | 5.5 | 0.1756 | 0.9812 | 0.0153 | 0.9388 |
| | histidine | NaCl 4.1% | | | | | |
| | | w/v | | | | | |
| | | mannitol | | | | | |
| 26 | 10 mM | 75 mM | 5.5 | 0.1899 | 0.9825 | 0.0188 | 0.9559 |
| | histidine | NaCl 3.3% | | | | | |
| | | w/v | | | | | |
| | | mannitol | | | | | |
| 27 | 5 mM | 9% w/v | 5.6 | 0.1776 | 0.9824 | 0.0164 | 0.9791 |
| | histidine | sucrose | | | | | |
| 28 | 5 mM | 9% w/v | 5.6 | 0.1726 | 0.9900 | 0.0163 | 0.9966 |
| | histidine | trehalose | | | | | |
| 29 | self- | 5% w/v | 5.4 | 0.2628 | 0.9972 | 0.0351 | 0.9531 |
| | buffered | mannitol | | | | | |

### Formulation Study B: Part I Results—Fragments

The CE-SDS reduced fragments values are shown in Table 14a and the CE-SDS reduced fragments values are shown in Table 14b. Both CE-SDS methods showed a time- and temperature-dependent increase in mirikizumab fragments. All formulations performed comparably to or better than Formulation 1.

TABLE 14a
CE-SDS reduced fragments at 5° C., 25° C. and 40° C.

| Formulation | Temp | 5° C. | | | 25° C. | | | 40° C. | | |
|---|---|---|---|---|---|---|---|---|---|---|
| No. | T = 0 | 4 w | 13 w | 26 w | 4 w | 8 w | 13 w | 2 w | 4 w | 8 w |
| 1 | 0.31 | 0.33 | 0.26 | 0.50 | 0.42 | 0.46 | 0.89 | 1.52 | 2.39 | 4.10 |
| 21 | 0.37 | 0.25 | 0.33 | 0.44 | 0.39 | 0.53 | 0.88 | 1.28 | 2.22 | 3.89 |
| 22 | 0.25 | 0.24 | 0.25 | 0.32 | 0.65 | 0.43 | 0.88 | 1.12 | 2.07 | 3.50 |
| 23 | 0.31 | 0.21 | 0.32 | 0.43 | 0.65 | 0.68 | 0.88 | 1.12 | 1.78 | 3.35 |
| 24 | 0.34 | 0.38 | | | 0.63 | 0.59 | 0.85 | 1.11 | 1.74 | 3.55 |
| 25 | 0.32 | 0.26 | 0.26 | 0.67 | 0.63 | 0.55 | 0.71 | 1.23 | 2.36 | 3.94 |
| 26 | 0.35 | 0.31 | 0.26 | 0.44 | 0.60 | 0.51 | 0.77 | 1.27 | 2.25 | 3.68 |
| 27 | 0.37 | 0.20 | 0.29 | 0.49 | 0.55 | 0.71 | 0.90 | 1.08 | 1.99 | 3.28 |
| 28 | 0.35 | 0.22 | 0.33 | 0.43 | 0.63 | 0.50 | 1.02 | 1.18 | 2.12 | 3.49 |
| 29 | 0.49 | 0.20 | 0.33 | 0.43 | 0.60 | 0.57 | 0.72 | 1.18 | 2.13 | 3.61 |

TABLE 14b
CE-SDS non-reduced fragments at 5° C., 25° C. and 40° C.

| Formulation | Temp | 5° C. | | | 25° C. | | | 40° C. | | |
|---|---|---|---|---|---|---|---|---|---|---|
| No. | T = 0 | 4 w | 13 w | 26 w | 4 w | 8 w | 13 w | 2 w | 4 w | 8 w |
| 1 | 1.26 | 1.31 | 1.10 | 1.31 | 1.48 | 1.51 | 1.83 | 2.40 | 3.65 | 4.50 |
| 21 | 1.32 | 1.26 | 1.14 | 1.27 | 1.64 | 1.44 | 1.84 | 2.37 | 5.45 | 4.09 |
| 22 | 1.41 | 1.31 | 1.06 | 1.14 | 2.02 | 1.49 | 1.71 | 2.34 | 3.37 | 3.85 |
| 23 | 1.31 | 1.31 | 1.03 | 1.25 | 1.90 | 1.48 | 1.67 | 2.20 | 3.05 | 3.68 |
| 24 | 1.32 | 1.41 | | | 1.50 | 1.40 | 1.62 | 2.24 | 3.33 | 3.86 |
| 25 | 1.30 | 1.34 | 1.08 | 1.25 | 1.77 | 1.51 | 1.74 | 2.31 | 3.19 | 3.96 |
| 26 | 1.25 | 1.34 | 1.06 | 1.20 | 1.54 | 1.55 | 1.66 | 2.28 | 3.67 | 3.64 |
| 27 | 1.30 | 1.40 | 1.13 | 1.45 | 1.56 | 1.42 | 1.73 | 2.21 | 3.77 | 3.93 |
| 28 | 1.19 | 1.62 | 1.10 | 1.25 | 1.58 | 1.54 | 1.79 | 2.24 | 3.70 | 4.02 |
| 29 | 1.38 | 1.35 | 1.08 | 1.24 | 1.79 | 1.64 | 1.80 | 2.55 | 3.96 | 5.02 |

### Formulation Study B: Part I Results—Charge Variants

icIEF main peak degradation rates at 25° C. and 40° C. are shown in Table 15. icIEF showed a time- and temperature-dependent decrease in mirikizumab charge variant main peak. This was largely attributable to acidic variant formation. A small (˜<2%) increase in basic variants was observed after 8 weeks at 40° C. All formulations performed comparably to Formulation 1. Formulations 1, 25 and 26 comprising sodium chloride appear to provide a benefit of slowing charge variant formation.

TABLE 15
icIEF Main Peak Degradation Rates at Elevated Temperature
(The data was fit to a simple linear regression to determine a degradation rate)

| | | | | Degradation | R | Degradation | R |
|---|---|---|---|---|---|---|---|
| Formulation | | | | Rate, 40° C. | squared, | Rate, 25° C. | squared, |
| No. | Buffer | Excipients | pH | (%/week) | 40° C. | (%/week) | 25° C. |
| 1 | 10 mM | 150 mM | 5.5 | −3.4663 | 0.9952 | −0.5270 | 0.9851 |
| | citrate | NaCl | | | | | |
| 21 | 5 mM | 5% w/v | 5.5 | −3.9506 | 0.9996 | −0.5156 | 0.9953 |
| | citrate | mannitol | | | | | |
| 22 | 5 mM | 5% w/v | 5.6 | −3.7945 | 0.9995 | −0.5915 | 0.9551 |
| | histidine | mannitol | | | | | |
| 23 | 5 mM | 5% w/v | 5.9 | −4.1399 | 0.9996 | −0.6522 | 0.9970 |
| | histidine | mannitol | | | | | |
| 24 | 5 mM | 5% w/v | 6.2 | −4.1803 | 1.0000 | −0.6558 | 0.9729 |
| | histidine | mannitol | | | | | |
| 25 | 10 mM | 37.5 mM | 5.5 | −3.7592 | 0.9966 | −0.7196 | 0.9860 |
| | histidine | NaCl 4.1% | | | | | |
| | | w/v | | | | | |
| | | mannitol | | | | | |
| 26 | 10 mM | 75 mM | 5.5 | −3.6310 | 0.9983 | −0.5482 | 0.9987 |
| | histidine | NaCl 3.3% | | | | | |
| | | w/v | | | | | |
| | | mannitol | | | | | |
| 27 | 5 mM | 9% w/v | 5.6 | −4.1677 | 0.9952 | −0.6874 | 0.9866 |
| | histidine | sucrose | | | | | |
| 28 | 5 mM | 9% w/v | 5.6 | −3.8439 | 0.9976 | −0.6724 | 0.9742 |
| | histidine | trehalose | | | | | |
| 29 | self- | 5% w/v | 5.4 | −3.7028 | 0.9962 | −0.6380 | 0.9664 |
| | buffered | mannitol | | | | | |

### Formulation Study B: Part I Results—Subvisible Particles

Subvisible particle data revealed that the ≥2 μm particle counts at 5° C. remained at ˜5000 particles/mL throughout the six months, except for Formulations 23 and 24, both of which exhibited refrigerated solubility issues). Samples stored at 25° C. and especially 40° C. consistently generated many more particles. Some formulations stored at elevated temperatures also showed a trend of increasing particle counts with increasing storage time.

### Formulation Study B: Part I Results—Viscosity and Glide Force

Viscosity is an important attribute of a drug formulation where the drug product is delivered by an enhanced prefilled syringe (ePFS) or auto-injector (AI) delivery system. As such, viscosities must be low enough to ensure that the AI device can achieve complete delivery of the dose and that, in the case of the ePFS, manual expulsion is not too difficult. The viscosities (at 15° C. and 20° C.) of the formulations prepared for Formulation Study B—Part I are shown in Table 16. The mirikizumab concentration is constant across the samples (˜125 mg/mL). Formulations 21-24 and 27-29 have a significantly higher viscosity compared to Formulation 1. Formulations 25 and 26, which contain NaCl and have a lower pH, have a viscosity that is only slightly higher than that of Formulation 1.

TABLE 16
Viscosity

| | | | | Viscosity | |
|---|---|---|---|---|---|
| Formu- | | | | (cP) | |
| lation | | | | 15° | 20° |
| No. | Buffer | Excipients | pH | C. | C. |
| 1 | 10 mM citrate | 150 mM NaCl | 5.5 | 8.2 | 6.4 |
| 21 | 5 mM citrate | 5% w/v mannitol | 5.5 | 15.4 | 11.7 |
| 22 | 5 mM histidine | 5% w/v mannitol | 5.6 | 15.6 | 11.9 |
| 23 | 5 mM histidine | 5% w/v mannitol | 5.9 | 17.5 | 13.3 |
| 24 | 5 mM histidine | 5% w/v mannitol | 6.2 | 18.1 | 13.7 |
| 25 | 10 mM histidine | 37.5 mM NaCl 4.1% | 5.5 | 10.4 | 8.1 |
| | | w/v mannitol | | | |
| 26 | 10 mM histidine | 75 mM NaCl 3.3% | 5.5 | 9.5 | 7.5 |
| | | w/v mannitol | | | |
| 27 | 5 mM histidine | 9% w/v sucrose | 5.6 | 17.2 | 13.1 |
| 28 | 5 mM histidine | 9% w/v trehalose | 5.6 | 18.1 | 13.7 |
| 29 | self-buffered | 5% w/v mannitol | 5.4 | 14.1 | 10.9 |

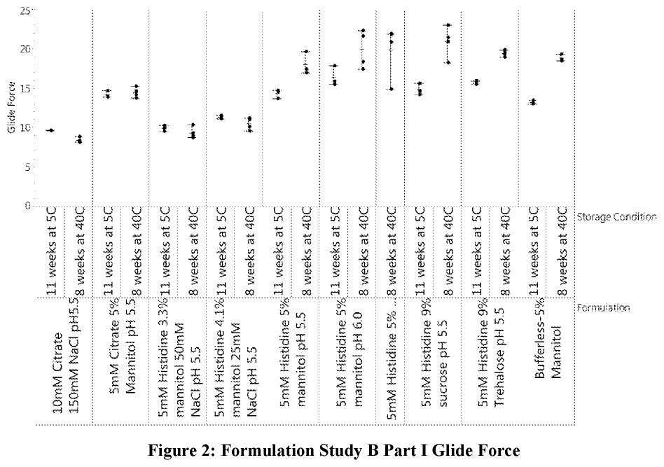

Glide force is another parameter that is helpful in differentiating between formulations. FIG. 2 illustrates that formulations from Formulation Study Part B demonstrate two distinct glide force profiles: those that do not change on accelerated stability and those that do. Removing ionic species such as NaCl from the formulation yields an increase in glide force. This change at accelerated conditions has ultimately manifested at 5° C. during long-term storage. This is possibly attributable to a gradual loss of silicone oil on the syringe barrel. Inclusion of ionic species ameliorates this loss of silicone oil and yields formulations that maintain consistent glide forces.

In view of the significantly higher viscosity of Formulations 21-24 and 27-29 and the impact on syringe glide force, replacing the citrate buffer and NaCl excipients to reduce injection site pain has to be balanced with the implications on viscosity and glide force. Accordingly, further formulations were designed and assessed in Formulation Study B: Part II.

### Formulation Study B: Part II Results—Purity

SEC, CE-SDS reduced and CE-SDS non-reduced monomer purity values for Formulations 1 and 30-36 at 5° C., 25° C. and 35° C. are shown in Tables 17a-17c. SEC and both CE-SDS methods showed a time- and temperature-dependent decrease in mirikizumab purity. All test formulations performed comparably to or better than Formulation 1. Formulations 30, 32 and 34 displayed the least decreases in purity at elevated temperatures over the course of the stability study.

TABLE 17a
SEC Monomer Purity at 5° C., 25° C. and 35° C.

| Formulation | Temp | 5° C. | | | | 25° C. | | | 35° C. | | |
|---|---|---|---|---|---|---|---|---|---|---|---|
| No. | T = 0 | 4 w | 8 w | 13 w | 26 w | 4 w | 8 w | 13 w | 2 w | 4 w | 8 w |
| 1 | 98.49 | | 98.36 | 98.46 | 98.25 | 98.28 | 98.05 | 98.04 | 98.15 | 97.86 | 97.22 |
| 30 | 98.43 | | 98.29 | | | 98.22 | 98.07 | 98.07 | 98.18 | 97.93 | 97.47 |
| 31 | 98.46 | | 98.36 | 98.48 | 98.23 | 98.15 | 98.03 | 98.03 | 98.14 | 97.72 | 97.20 |
| 32 | 98.66 | | 98.65 | 98.78 | 98.57 | 98.54 | 98.41 | 98.39 | 98.49 | 98.13 | 97.61 |
| 33 | 98.25 | | | | | 97.90 | | | 97.87 | 97.66 | |
| 34 | 98.39 | | 98.62 | 98.30 | 98.34 | 98.23 | 98.43 | 98.02 | 98.21 | 97.87 | 97.87 |
| 35 | 98.41 | 98.19 | | | | 98.04 | 97.97 | 97.86 | 98.02 | 97.60 | 97.22 |
| 36 | 98.37 | 98.06 | | | | 97.82 | 97.70 | 97.57 | 97.81 | 97.44 | 97.05 |

TABLE 17b
CE-SDS Reduced Monomer Purity at 5° C., 25° C. and 35° C.

| Formulation | Temp | 5° C. | | | | 25° C. | | | 35° C. | | |
|---|---|---|---|---|---|---|---|---|---|---|---|
| No. | T = 0 | 4 w | 8 w | 13 w | 26 w | 4 w | 8 w | 13 w | 2 w | 4 w | 8 w |
| 1 | 98.74 | | 98.83 | 98.80 | 98.82 | 98.52 | 98.20 | 98.19 | 98.40 | 97.79 | 96.83 |
| 30 | 98.86 | | 98.84 | | | 98.64 | 98.29 | 98.06 | 98.49 | 97.91 | 97.24 |
| 31 | 99.02 | | 98.92 | 98.77 | 98.82 | 98.49 | 98.21 | 98.17 | 98.51 | 97.86 | 97.35 |
| 32 | 98.78 | | 99.13 | 98.66 | 98.81 | 98.47 | 98.34 | 98.05 | 98.48 | 97.72 | 97.23 |
| 33 | 98.77 | | | | | 98.42 | | | 98.51 | 97.66 | |
| 34 | 99.12 | | 99.07 | 98.87 | 98.55 | 98.58 | 98.40 | 98.28 | 98.13 | 97.63 | 97.23 |
| 35 | 98.62 | 98.79 | | | | 98.67 | 98.36 | 97.96 | 98.35 | 97.76 | 96.89 |
| 36 | 98.58 | 98.91 | | | | 98.42 | 98.32 | 98.09 | 98.13 | 97.61 | 97.02 |

TABLE 17c
CE-SDS Non-Reduced Monomer Purity at 5° C., 25° C. and 35° C.

| Formulation | Temp | 5° C. | | | | 25° C. | | | 35° C. | | |
|---|---|---|---|---|---|---|---|---|---|---|---|
| No. | T = 0 | 4 w | 8 w | 13 w | 26 w | 4 w | 8 w | 13 w | 2 w | 4 w | 8 w |
| 1 | 98.59 | | 98.37 | 98.10 | 98.03 | 97.81 | 97.64 | 97.16 | 97.17 | 96.57 | 95.78 |
| 30 | 98.58 | | 98.26 | | | 97.75 | 97.56 | 97.26 | 97.25 | 96.88 | 96.42 |
| 31 | 98.51 | | 98.29 | 98.05 | 98.00 | 97.78 | 97.58 | 97.01 | 97.12 | 96.72 | 95.65 |
| 32 | 98.44 | | 98.48 | 98.24 | 98.15 | 97.93 | 97.81 | 97.28 | 97.39 | 96.86 | 96.20 |
| 33 | 98.24 | | | | | 97.53 | | | 96.93 | 96.55 | |
| 34 | 98.34 | | 98.22 | 98.43 | 98.38 | 97.93 | 97.23 | 97.59 | 97.56 | 96.86 | 95.93 |
| 35 | 97.91 | 98.33 | | | | 97.94 | 97.70 | 96.96 | 97.62 | 97.12 | 95.60 |
| 36 | 97.87 | 98.09 | | | | 97.59 | 97.43 | 96.51 | 97.44 | 96.81 | 95.43 |

### Formulation Study B: Part II Results—Aggregates

SEC total aggregates values for Formulations 1 and 30-36 at 5° C., 25° C. and 35° C. are shown in Table 18. SEC showed a time- and temperature-dependent increase in mirikizumab aggregates. All formulations performed comparably to Formulation 1.

Formulations 30, 32, and 34 displayed the smallest increases in aggregates over the course of the stability study.

TABLE 18
SEC Total Aggregates at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 1.51 | | 1.64 | 1.54 | 1.71 | 1.67 | 1.89 | 1.88 | 1.78 | 1.98 | 2.57 |
| 30 | 1.57 | | 1.71 | | | 1.73 | 1.91 | 1.91 | 1.78 | 1.95 | 2.33 |
| 31 | 1.54 | | 1.64 | 1.52 | 1.69 | 1.68 | 1.91 | 1.89 | 1.79 | 2.01 | 2.57 |
| 32 | 1.34 | | 1.35 | 1.22 | 1.37 | 1.34 | 1.53 | 1.50 | 1.43 | 1.62 | 2.09 |
| 33 | 1.75 | | | | | 2.04 | | | 2.10 | 2.28 | |
| 34 | 1.59 | | 1.38 | 1.62 | 1.60 | 1.67 | 1.47 | 1.86 | 1.66 | 1.93 | 1.82 |
| 35 | 1.50 | 1.72 | | | | 1.85 | 1.99 | 2.07 | 1.85 | 2.21 | 2.63 |
| 36 | 1.55 | 1.85 | | | | 2.12 | 2.28 | 2.39 | 2.17 | 2.50 | 2.91 |

### Formulation Study B: Part II Results—Fragments

CE-SDS Reduced and CE-SDS Non-Reduced fragment values for Formulations 1 and 30-36 at 5° C., 25° C. and 35° C. are shown in Tables 19a and 19b. Both CE-SDS methods showed a time- and temperature-dependent increase in mirikizumab fragments. All formulations performed comparably to or better than Formulation 1.

TABLE 19a
CE-SDS Reduced Fragments at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 0.20 | | 0.31 | 0.39 | 0.42 | 0.59 | 0.93 | 0.86 | 0.79 | 1.38 | 2.20 |
| 30 | 0.26 | | 0.18 | | | 0.51 | 0.79 | 0.84 | 0.57 | 1.17 | 1.59 |
| 31 | 0.19 | | 0.18 | 0.41 | 0.43 | 0.64 | 0.84 | 0.89 | 0.72 | 1.20 | 1.78 |
| 32 | 0.29 | | 0.17 | 0.45 | 0.49 | 0.64 | 0.86 | 0.97 | 0.79 | 1.39 | 1.93 |
| 33 | 0.35 | | | | | 0.63 | | | 0.55 | 1.19 | |
| 34 | 0.19 | | 0.26 | 0.45 | 0.78 | 0.51 | 0.80 | 0.93 | 0.98 | 1.39 | 1.86 |
| 35 | 0.50 | 0.35 | | | | 0.50 | 0.85 | 0.94 | 0.92 | 1.33 | 2.04 |
| 36 | 0.52 | 0.31 | | | | 0.54 | 0.77 | 0.78 | 0.82 | 1.18 | 2.05 |

TABLE 19b
CE-SDS Non-Reduced Fragments at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 1.10 | | 1.19 | 1.39 | 1.48 | 1.63 | 1.74 | 2.05 | 2.05 | 2.56 | 3.28 |
| 30 | 1.00 | | 1.17 | | | 1.53 | 1.72 | 1.82 | 1.98 | 2.20 | 2.58 |
| 31 | 1.11 | | 1.25 | 1.46 | 1.47 | 1.62 | 1.72 | 2.14 | 2.20 | 2.45 | 3.38 |
| 32 | 1.27 | | 1.21 | 1.38 | 1.48 | 1.62 | 1.71 | 2.14 | 2.13 | 2.53 | 3.10 |
| 33 | 1.18 | | | | | 1.53 | | | 2.08 | 2.20 | |
| 34 | 1.30 | | 1.34 | 1.22 | 1.22 | 1.54 | 1.84 | 1.84 | 1.82 | 2.43 | 3.03 |
| 35 | 1.59 | 1.13 | | | | 1.38 | 1.71 | 2.12 | 1.71 | 2.06 | 3.25 |
| 36 | 1.44 | 1.16 | | | | 1.41 | 1.64 | 2.07 | 1.57 | 1.95 | 2.96 |

### Formulation Study B: Part II Results—Charge Variants

icIEF charge variant main peak values for Formulations 1 and 30-36 at 5° C., 25° C. and 35° C. are shown in Table 20a. Total acidic variant values for Formulations 1 and 30-36 at 5° C., 25° C. and 35° C. are shown in Table 20b. Total basic variant values for Formulations 1 and 30-36 at 5° C., 25° C. and 35° C. are shown in Table 20c.

icIEF showed a time- and temperature-dependent decrease in mirikizumab charge variant main peak. This was largely attributable to acidic variant formation. A small (˜<2%) increase in basic variants was observed after 8 weeks at 35° C. All formulations performed comparably to Formulation 1.

TABLE 20a
icIEF Main Peak at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 74.53 | | 76.96 | 77.54 | 76.64 | 74.09 | 72.90 | 71.71 | 72.53 | 67.30 | 60.15 |
| 30 | 75.32 | | 76.28 | | | 74.10 | 72.57 | 71.67 | 72.20 | 66.31 | 58.65 |
| 31 | 74.59 | | 77.08 | 77.40 | 76.93 | 74.20 | 71.70 | 72.19 | 73.94 | 66.67 | 60.44 |
| 32 | 75.71 | | 77.65 | 78.37 | 77.86 | 73.90 | 71.12 | 71.26 | 73.12 | 67.46 | 60.52 |
| 33 | 74.51 | | | | | 73.55 | | | 71.32 | 65.08 | |
| 34 | 77.29 | | 73.28 | 76.31 | 74.13 | 72.40 | 71.84 | 70.92 | 71.52 | 64.76 | 59.85 |
| 35 | 75.77 | 75.66 | | | | 74.25 | 73.62 | 73.12 | 71.86 | 68.53 | 62.29 |
| 36 | 75.57 | 75.53 | | | | 73.93 | 71.87 | 71.32 | 70.91 | 66.89 | 59.73 |

TABLE 20b
Total Acidic Variants at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 22.47 | | 21.39 | 20.76 | 21.95 | 23.12 | 24.90 | 25.81 | 25.08 | 29.06 | 36.68 |
| 30 | 21.75 | | 22.13 | | | 23.25 | 25.31 | 26.09 | 25.63 | 30.18 | 38.93 |
| 31 | 22.47 | | 21.17 | 20.91 | 21.55 | 22.77 | 25.72 | 25.17 | 23.64 | 28.94 | 35.97 |
| 32 | 21.61 | | 20.71 | 20.11 | 20.57 | 23.00 | 25.74 | 25.80 | 24.21 | 28.21 | 35.66 |
| 33 | 22.58 | | | | | 24.14 | | | 26.54 | 32.28 | |
| 34 | 21.56 | | 23.72 | 22.20 | 23.77 | 24.89 | 25.37 | 26.54 | 25.09 | 31.90 | 36.51 |
| 35 | 21.75 | 22.02 | | | | 23.04 | 24.40 | 24.71 | 25.08 | 27.55 | 34.73 |
| 36 | 21.82 | 22.18 | | | | 23.59 | 26.25 | 26.77 | 26.61 | 29.92 | 38.10 |

TABLE 20b
Total Basic Variants at 5° C., 25° C. and 35° C.

| Formu- | | | | | | | | | | | |
|---|---|---|---|---|---|---|---|---|---|---|---|
| lation | Temp | 5° C. | | | | 25° C. | | | 35° C. | | |
| No. | T = 0 | 4 w | 8 w | 13 w | 26 w | 4 w | 8 w | 13 w | 2 w | 4 w | 8 w |
| 1 | 3.01 | | 1.65 | 1.70 | 1.42 | 2.80 | 2.20 | 2.47 | 2.39 | 3.65 | 3.17 |
| 30 | 2.93 | | 1.58 | | | 2.65 | 2.12 | 2.24 | 2.17 | 3.51 | 2.42 |
| 31 | 2.94 | | 1.75 | 1.69 | 1.52 | 3.03 | 2.59 | 2.64 | 2.42 | 4.40 | 3.59 |
| 32 | 2.68 | | 1.63 | 1.52 | 1.57 | 3.10 | 3.14 | 2.94 | 2.66 | 4.33 | 3.82 |
| 33 | 2.91 | | | | | 2.31 | | | 2.13 | 2.64 | |
| 34 | 1.15 | | 3.00 | 1.49 | 2.09 | 2.71 | 2.79 | 2.54 | 3.39 | 3.34 | 3.63 |
| 35 | 2.47 | 2.33 | | | | 2.71 | 1.98 | 2.17 | 3.06 | 3.92 | 2.98 |
| 36 | 2.61 | 2.29 | | | | 2.48 | 1.88 | 1.91 | 2.48 | 3.19 | 2.16 |

### Formulation Study B: Part II Results—Viscosity

The viscosities (at 15° C. and 20° C.) of the formulations prepared for Formulation Study B—Part II are shown in Table 21. The mirikizumab concentration is roughly constant across the samples (˜125 mg/mL). It was observed in Formulation Study B Part I and confirmed in this study that elimination or reduction in the concentration of NaCl leads to increased viscosity. The data in Table 21 illustrates that reduction of the pH can lower viscosity.

TABLE 21
Viscosity

| | | | | Viscosity | |
|---|---|---|---|---|---|
| Formu- | | | | (cP) | |
| lation | | | | 15° | 20° |
| No. | Buffer | Excipients | pH | C. | C. |
| 1 | 10 mM citrate | 150 mM NaCl | 5.4 | 8.3 | 6.5 |
| 30 | 5 mM histidine | 25 mM NaCl 4.1% | 5.9 | 12.6 | 9.6 |
| | | w/v mannitol | | | |
| 31 | self-buffered | 25 mM NaCl 4.1% | 5.3 | 11.3 | 8.6 |
| | | w/v mannitol | | | |
| 32 | 5 mM histidine | 25 mM NaCl 4.1% | 5.2 | 9.9 | 7.6 |
| | | w/v mannitol | | | |
| 33 | 5 mM histidine | 25 mM NaCl 4.1% | 6.3 | NT | NT |
| | | w/v mannitol | | | |
| 34 | 5 mM histidine | 25 mM NaCl 4.1% | 5.6 | 11.1 | 8.5 |
| | | w/v mannitol | | | |
| 35 | self-buffered | 150 mM NaCl | 5.5 | 7.2 | 5.8 |
| 36 | self-buffered | 25 mM NaCl 4.1% | 6.0 | 12.1 | 9.7 |
| | | w/v mannitol | | | |

The data from Formulation Study B Parts I and II was assessed and preferred formulations were designed and assessed in Formulation Study B Part III.

### Formulation Study B: Part III Results—Purity

SEC, CE-SDS Reduced and CE-SDS Non-Reduced monomer purity values for Formulations 1 and 37-40 at 5° C., 25° C. and 35° C. are shown in Tables 22a-22c. SEC and both CE-SDS methods showed a time- and temperature-dependent decrease in mirikizumab purity. All test formulations performed comparably to or better than Formulation 1.

TABLE 22a
SEC Monomer Purity at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 98.80 | 98.81 | 98.83 | 98.78 | 98.57 | 98.49 | 98.28 | 98.47 | 98.18 | 97.63 |
| 37 | 99.17 | 99.07 | 99.07 | 99.05 | 98.82 | 98.72 | 98.61 | 98.71 | 98.44 | 97.89 |
| 38 | 98.63 | 98.57 | 98.57 | 98.55 | 98.35 | 98.27 | 98.04 | 98.25 | 97.96 | 97.36 |
| 39 | 98.51 | 98.43 | 98.34 | 98.51 | 98.29 | 98.16 | 98.06 | | 97.89 | 97.44 |
| 40 | 99.31 | 99.24 | 99.30 | 99.19 | 99.04 | 98.69 | 98.64 | 98.86 | 98.47 | 98.05 |

TABLE 22b
CE-SDS Reduced Monomer Purity at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 98.79 | 98.66 | 98.62 | 98.92 | 98.41 | 98.53 | 98.14 | 98.34 | 97.28 | 97.19 |
| 37 | 98.46 | 98.72 | 98.77 | 99.07 | 98.72 | 98.51 | 98.13 | 98.52 | 98.10 | 97.52 |
| 38 | 98.90 | 98.92 | 98.73 | 98.80 | 98.37 | 98.50 | 97.94 | 98.46 | 98.00 | 97.50 |
| 39 | 99.10 | 98.64 | 98.67 | 98.98 | 98.64 | 98.31 | 98.21 | | 98.03 | 97.05 |
| 40 | 98.75 | 98.82 | 98.99 | 98.90 | 98.71 | 98.69 | 97.91 | 98.30 | 98.12 | 97.51 |

TABLE 22c
CE-SDS Non-Reduced Monomer Purity at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 98.12 | 98.48 | 97.04 | 98.22 | 98.17 | 97.41 | 97.12 | 97.54 | 97.26 | 95.59 |
| 37 | 98.24 | 98.53 | 98.09 | 98.27 | 98.22 | 97.43 | 97.25 | 97.51 | 97.48 | 95.80 |
| 38 | 97.86 | 98.50 | 98.00 | 98.09 | 98.13 | 97.35 | 97.02 | 97.53 | 97.27 | 95.40 |
| 39 | 98.39 | 98.19 | 98.11 | 98.36 | 97.77 | 97.43 | 97.04 | | 97.06 | 96.12 |
| 40 | 98.49 | 98.29 | 97.89 | 98.29 | 97.84 | 97.50 | | 97.96 | 96.94 | 95.84 |

### Formulation Study B: Part III Results—Aggregates

SEC total aggregates values for Formulations 1 and 37-40 at 5° C., 25° C. and 35° C. are shown in Table 23. SEC showed a time- and temperature-dependent increase in mirikizumab aggregates. All formulations performed comparably to Formulation 1.

TABLE 23
SEC Total Aggregates at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 1.17 | 1.19 | 1.17 | 1.22 | 1.36 | 1.46 | 1.62 | 1.46 | 1.69 | 2.17 |
| 37 | 0.83 | 0.92 | 0.93 | 0.95 | 1.09 | 1.19 | 1.31 | 1.22 | 1.46 | 1.88 |
| 38 | 1.37 | 1.42 | 1.40 | 1.45 | 1.58 | 1.66 | 1.86 | 1.67 | 1.92 | 2.37 |
| 39 | 1.49 | 1.55 | 1.60 | 1.49 | 1.61 | 1.76 | 1.86 | | 1.90 | 2.33 |
| 40 | 0.69 | 0.76 | 0.70 | 0.81 | 0.92 | 1.17 | 1.20 | 1.05 | 1.32 | 1.80 |

### Formulation Study B: Part III Results—Fragments

CE-SDS reduced and CE-SDS non-reduced fragment values for Formulations 1 and 37-40 at 5° C., 25° C. and 35° C. are shown in Tables 24a and 24b. Both CE-SDS methods showed a time- and temperature-dependent increase in mirikizumab fragments. All formulations performed comparably to or better than Formulation 1.

TABLE 24a
CE-SDS Reduced Fragments at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 0.41 | 0.57 | 0.47 | 0.33 | 0.74 | 0.62 | 0.96 | 0.76 | 1.68 | 1.98 |
| 37 | 0.67 | 0.57 | 0.48 | 0.23 | 0.51 | 0.62 | 0.97 | 0.63 | 1.03 | 1.53 |
| 38 | 0.42 | 0.40 | 0.61 | 0.35 | 0.69 | 0.70 | 0.99 | 0.70 | 1.11 | 1.67 |
| 39 | 0.23 | 0.50 | 0.52 | 0.27 | 0.53 | 0.93 | 1.02 | | 1.15 | 2.00 |
| 40 | 0.59 | 0.35 | 0.26 | 0.27 | 0.45 | 0.44 | 1.13 | 0.76 | 0.94 | 1.60 |

TABLE 24b
CE-SDS Non-Reduced Fragments at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 1.57 | 1.25 | 2.58 | 1.29 | 1.43 | 2.01 | 2.20 | 1.85 | 2.11 | 3.37 |
| 37 | 1.38 | 1.19 | 1.51 | 1.24 | 1.40 | 1.93 | 2.05 | 1.83 | 1.95 | 3.17 |
| 38 | 1.68 | 1.21 | 1.53 | 1.34 | 1.41 | 2.00 | 2.26 | 1.80 | 2.12 | 3.49 |
| 39 | 1.25 | 1.52 | 1.61 | 1.28 | 1.82 | 2.11 | 2.35 | | 2.39 | 3.11 |
| 40 | 1.24 | 1.43 | 1.74 | 1.31 | 1.74 | 1.98 | | 1.59 | 2.41 | 3.33 |

### Formulation Study B: Part III Results—Charge Variants

icIEF charge variant main peak values for Formulations 1 and 37-40 at 5° C., 25° C. and 35° C. are shown in Table 25a. Total acidic variant values for Formulations 1 and 37-40 at 5° C., 25° C. and 35° C. are shown in Table 25b. Total basic variant values for Formulations 1 and 37-40 at 5° C., 25° C. and 35° C. are shown in Table 25c.

icIEF showed a time- and temperature-dependent decrease in mirikizumab charge variant main peak. This was largely attributable to acidic variant formation. A small (˜<2%) increase in basic variants was observed after 8 weeks at 35° C. All formulations performed comparably to Formulation 1.

TABLE 25a
icIEF Main Peak at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 77.78 | 76.89 | 77.20 | 77.01 | 75.35 | 73.19 | 72.21 | 72.62 | 67.51 | 60.38 |
| 37 | 78.27 | 78.72 | 78.66 | 79.48 | 76.72 | 76.23 | 74.26 | 74.36 | 70.03 | 63.17 |
| 38 | 77.23 | 76.57 | 77.36 | 77.13 | 74.34 | 74.56 | 71.57 | 74.53 | 69.12 | 62.48 |
| 39 | 78.34 | 76.03 | 78.06 | 78.25 | 74.08 | 74.84 | 71.38 | | 71.43 | 62.26 |
| 40 | 77.29 | 78.68 | 79.40 | 78.51 | 77.38 | 75.52 | 73.06 | 71.02 | 69.74 | 62.27 |

TABLE 25b
Total Acidic Variants at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 20.71 | 21.73 | 21.23 | 20.57 | 23.02 | 24.30 | 25.17 | 24.97 | 30.05 | 35.84 |
| 37 | 19.33 | 19.31 | 19.20 | 18.01 | 20.87 | 20.80 | 22.58 | 21.95 | 27.09 | 32.66 |
| 38 | 20.63 | 21.74 | 21.01 | 20.66 | 23.60 | 22.95 | 25.72 | 22.88 | 28.40 | 33.63 |
| 39 | 18.11 | 22.11 | 19.03 | 18.13 | 23.35 | 21.93 | 24.34 | | 24.89 | 33.12 |
| 40 | 19.08 | 18.44 | 18.70 | 18.72 | 19.89 | 21.43 | 23.76 | 25.92 | 25.76 | 33.52 |

TABLE 25c
Total Basic Variants at 5° C., 25° C. and 35° C.

| Formu- | | | | | | | | | | |
|---|---|---|---|---|---|---|---|---|---|---|
| lation | Temp | 5° C. | | | 25° C. | | | 35° C. | | |
| No. | T = 0 | 4 w | 12 w | 26 w | 4 w | 8 w | 12 w | 2 w | 4 w | 8 w |
| 1 | 1.51 | 1.38 | 1.57 | 2.42 | 1.63 | 2.51 | 2.62 | 2.41 | 2.44 | 3.78 |
| 37 | 2.40 | 1.97 | 2.14 | 2.52 | 2.40 | 2.97 | 3.15 | 3.69 | 2.88 | 4.18 |
| 38 | 2.14 | 1.69 | 1.63 | 2.21 | 2.06 | 2.48 | 2.71 | 2.60 | 2.48 | 3.88 |
| 39 | 3.55 | 1.85 | 2.91 | 3.62 | 2.56 | 3.23 | 4.28 | | 3.68 | 4.62 |
| 40 | 3.63 | 2.88 | 1.90 | 2.77 | 2.73 | 3.05 | 3.18 | 3.06 | 4.50 | 4.21 |

### Formulation Study B: Conclusions

The purpose of Formulation Study B was to identify a high concentration mirikizumab formulation that may reduce injection pain discomfort that may be associated with formulations comprising NaCl and/or citrate buffer while maintaining the excellent stability characteristics of the preferred formulations identified in Formulation Study Part A. Through the series of studies described above, the preferred formulation comprises (i) mirikizumab, (ii) 5 mM of a histidine buffer, (iii) 50 mM of NaCl, (iv)3.3% w/v of mannitol, and (v) 0.03% w/v of polysorbate 80, wherein the pH of the formulation is 5.5.

The formulations described herein may be evaluated in clinical trials in human patients.

### Example 4: Clinical Study—Assessment of Mirikizumab Formulations in Healthy Subjects

### Overview

The preferred formulation from Formulation Study A (mirikizumab, 10 mM citrate buffer, 150 mM NaCl, 0.05% w/v polysorbate 80, pH 5.5)(hereinafter referred to as Formulation A-P) and the preferred formulation from Formulation Study B (mirikizumab, 5 mM of a histidine buffer, 50 mM of NaCl, 3.3% w/v of mannitol, 0.03% w/v of polysorbate 80, pH 5.5)(Formulation B-P) were investigated in clinical trials in human patients to compare relative bioavailability and injection site reaction profiles, in particular, injection site pain profiles.

The study is a Phase 1, subject-blind, investigator-blind, 2-arm, randomized, single dose, parallel design study in healthy subjects. Eligible subjects were admitted to the clinical research unit (CRU) on Day −1 and randomized 1:1 to 1 of 2 possible treatments and, within treatments, 1:1:1 to 3 possible injection locations (arms, thighs, or abdomen) using a computer-generated allocation code. Subjects were allowed to leave the CRU after completing the 4-hour safety assessments on Day 1, at the investigator's discretion, and were to return for pharmacokinetic sampling and safety assessments at predefined outpatient visits up to 12 weeks post dose. Safety and tolerability were assessed from clinical laboratory tests, vital sign measurements, recording of adverse events and physical examination.

Formulation A-P and Formulation B-P were as 1-mL single-dose, pre-filled, disposable manual syringes designed to deliver 100 mg of mirikizumab. The study duration for each participant was up to 16 weeks, which included a 4-week screening period, intervention on Day 1, and 12 week post-dose assessment period with follow-up. On Day 1, subjects received 2×1-mL PFS subcutaneous (SC) injections into the arms, thighs, or abdomen, according to the randomization schedule.

### Objectives

Certain objectives of the study are:
i) To evaluate the relative bioavailability of a single 200-mg SC dose (2×1-mL PFS injections) of mirikizumab Formulation B-P compared to the mirikizumab Formulation A-P
   The endpoints are Cₘₐₓ, AUC(0-∞), and AUC(0-tₗₐₛₜ) (AUC(0-00)=area under the concentration versus time curve from time 0 to infinity; AUC(0tₗₐₛₜ)=area under the concentration versus time curve from time zero to time t, where t is the last time point with a measurable concentration; Cₘₐₓ=maximum observed drug concentration).
ii) To evaluate the safety and tolerability of a single 200-mg SC dose (2×1-mL PFS injections) of mirikizumab Formulation B-P compared to the mirikizumab Formulation A-P;
   The endpoints are Treatment Emergent Adverse Effects (TEAEs) and Serious Adverse Effects (SAEs).
iii) To evaluate injection site reactions (ISRs), including pain
   The endpoints are severity, duration and location of erythema, bruising, induration, pain, pruritus, and edema, and the VAS pain score and bleeding immediately after injection.

### Methods

Subjects were required to be overtly healthy males or females, aged between 18 and 75 years, with a body mass index of 18.0 to 32.0 kg/m2, inclusive, at screening. Of the 60 subjects enrolled in the study, 19 were male and 41 were female. The subjects' age ranged from 19 to 74 years.

Mirikizumab Formulation A-P and mirikizumab Formulation B-P were supplied as 1-mL single-dose, pre-filled, disposable manual syringes designed to deliver 100 mg of mirikizumab.

On Day 1, subjects received 2×1-mL PFS SC injections into the arms, thighs, or abdomen.

Subjects randomized to a group with the arm or thigh as the injection area will have:
(a) the first injection administered to the left limb, and
(b) the second injection administered to the corresponding (contra-lateral) right limb.

Subjects randomized to the group with the abdomen as the injection area will have
(a) the first injection administered to the lower left quadrant, and
(b) the second injection administered to the lower right quadrant of the abdomen. The second injection should be administered 20 (±2) minutes after the first injection.

Outpatient visits occurred on Days 3, 5, 8, 11, 15, 22, 29, 43, 57, 71 and 85. Pharmacokinetic (PK) samples were collected on Days 1 (pre-dose), 3, 5, 8, 11, 15, 22, 29, 43, 57, 71 and 85. AE and concomitant medication assessments were performed on Days −1, 1, 3, 5, 8, 11, 15, 22, 29, 36, 43, 50, 57, 64, 71 and 85. Safety assessment telephone calls were performed on Days 36, 50 and 64. Injection site assessments for erythema, induration, pruritus, edema, pain (first injection site only), and bruising were performed at 1, 5, 15, 30, 60, 120 and 240 minutes post-dose on Day 1.

### Results

(a) Pharmacokinetic analyses

The following PK parameter estimates for mirikizumab were calculated using noncompartmental methods using Phoenix WinNonlin Version 8.1.

| Parameter | Units | Definition |
|---|---|---|
| AUC(0-tₗₐₛₜ) | day*μg/mL | area under the concentration versus |
| | | time curve from time zero to time t, |
| | | where t is the last time point with a |
| | | measurable concentration |
| AUC(0-∞) | day*μg/mL | area under the concentration versus time |
| | | curve from time zero to infinity |
| % AUC(tₗₐₛₜ-∞) | % | percentage of AUC(0-∞) extrapolated |
| Cₘₐₓ | μg/mL | maximum observed drug concentration |
| tₘₐₓ | day | time of maximum observed drug |
| | | concentration |
| t_{1/2} | day | half-life associated with the terminal |
| | | rate constant (λz) in non- |
| | | compartmental analysis |
| CL/F | L/day | apparent total body clearance of drug |
| | | calculated after extravascular |
| | | administration |
| V_{z}/F | L | apparent volume of distribution during |
| | | the terminal phase after extravascular |
| | | administration |
| Vₛₛ/F | L | apparent volume of distribution at |
| | | steady state after extravascular |
| | | administration |

Arithmetic mean concentration-time profiles were plotted using nominal time points per the protocol. Mean concentrations were plotted for a given time if ⅔ of the individual data at that time point had quantifiable measurements within the sampling window (±10%).

Statistical analysis of the PK parameters between mirikizumab Formulation A-P and mirikizumab Formulation B-P. Log-transformed Cₘₐₓ, AUC(0tₗₐₛₜ) and AUC(0-∞) parameters were evaluated in a linear fixed effects model with fixed effects for treatment formulation and injection-site location. The differences between the mirikizumab Formulation A-P and mirikizumab Formulation B-P were back-transformed to present the ratios of geometric LS means and the corresponding 90% CI. Parameters were summarized by treatment formulation.

The summary PK parameters for mirikizumab Formulation A-P and mirikizumab Formulation B-P are shown in Table 26.

TABLE 26
Summary of the Pharmacokinetic Parameters of Mirikizumab

| | Geometric mean (Geometric CV %) [n] | | | |
|---|---|---|---|---|
| | Formulation A-P | | Formulation B-P | |
| Parameter | (N = 30) | | (N = 30) | |
| AUC(0-tlast) (ug · day/mL) | 225 | (56%) [30] | 206 | (46%) [30] |
| AUC(0-∞) (ug · day/mL) | 229 | (56%) [30] | 209 | (45%) [30] |
| % AUC(tlast-∞) (%) | 1.52 | (59%) [30] | 1.59 | (61%) [30] |
| Cmax (ug/mL) | 12.7 | (48%) [30] | 11.6 | (45%) [30] |
| tmax (day)# | 4.00 | (4.00-7.00) [30] | 4.00 | (2.00-10.00) [30] |
| t½ (day)* | 11.5 | (6.56-18.7) [30] | 11.8 | (7.53-17.4) [30] |
| CL/F (L/day) | 0.874 | (56%) [30] | 0.955 | (45%) [30] |
| Vz/F (L) | 14.5 | (40%) [30] | 16.3 | (43%) [30] |
| Vss/F (L) | 15.4 | (44%) [30] | 17.1 | (47%) [30] |

Abbreviations: % AUC(tₗₐₛₜ-∞) = percentage of AUC(0-∞) extrapolated;
AUC(0-∞) = area under the concentration versus time curve from time zero to infinity;
AUC(0-tlast) = area under the concentration versus time curve from time zero to time t, where t is is the last time point with a measurable concentration;
CL/F = apparent total body clearance calculated after extravascular administration;
Cmax = maximum observed drug concentration;
CV = coefficient of variation;
N = number of subjects;
n = number of observations;
t½ = half-life associated with the terminal rate constant in noncompartmental analysis;
tmax = time of maximum observed drug concentration;
Vss/F = apparent volume of distribution at steady state after extravascular administration;
Vz/F = apparent volume of distribution during the terminal phase after extravascular administration
#Median (minimum-maximum)
*Geometric mean (minimum-maximum)
Formulation A-P = 200 mg Mirikizumab Formulation (100 mg/mL) 2 × 1 mL PFS;
Formulation B-P = 200 mg Mirikizumab Formulation (100 mg/mL) 2 × 1 mL PFS

Overall, no statistically significant differences in Cₘₐₓ, AUC(0 ∞), and AUC(0 tlast) were observed following administration of the mirikizumab Formulation A-P and mirikizumab Formulation B-P, with the 90% CIs for the ratios of geometric LS means including unity (Table 27).

TABLE 27
Statistical Analysis of the Pharmacokinetic Parameters of Mirikizumab

| | | | | Ratio of | |
|---|---|---|---|---|---|
| | | | Geometric | geometric | 90% CI |
| | | | least | least squares | for the |
| | | | squares | mean (Formulation | ratio |
| Parameter | Treatment | n | mean | B-P:Formulation A-P) | (Lower, Upper) |
| AUC(0-tlast) | Formulation | 30 | 225 | | |
| (ug · day/mL) | A-P | | | | |
| | Formulation | 30 | 206 | 0.915 | (0.760, 1.10) |
| | B-P | | | | |
| AUC(0-∞) | Formulation | 30 | 229 | | |
| (ug · day/mL) | A-P | | | | |
| | Formulation | 30 | 209 | 0.915 | (0.761, 1.10) |
| | B-P | | | | |
| Cmax (ug/mL) | Formulation | 30 | 12.7 | | |
| | A-P | | | | |
| | Formulation | 30 | 11.6 | 0.907 | (0.775, 1.06) |
| | B-P | | | | |

Abbreviations: AUC(0-∞) = area under the concentration versus time curve from time zero to infinity;
AUC(0-tₗₐₛₜ) = area under the concentration versus time curve from time zero to time t, where t is the last time point with a measurable concentration;
CI = confidence interval;
Cmax = maximum observed drug concentration;
n = number of observations
Formulation A-P = 200 mg Mirikizumab Formulation (100 mg/mL) 2 × 1 mL PFS;
Formulation B-P = 200 mg Mirikizumab Formulation (100 mg/mL) 2 × 1 mL PFSModel: Log(PK) = Treatment + Location + Random Error

There was no statistically significant difference in the median tmax of mirikizumab between the formulations. Serum concentrations of mirikizumab declined after tmax, and the resulting geometric mean t½ values following dosing with the mirikizumab Formulation A-P and mirikizumab Formulation B-P were similar, being 11.5 days (276 hours) and 11.8 days (283 hours), respectively. Between subject variability (CV %) estimates for AUC(0-tₗₐₛₜ), AUC(0 co), and Cₘₐₓ were moderate to high 48% to 56% for the mirikizumab Formulation A-P, and 45% to 46% for the mirikizumab Formulation B-P.

### (b) Safety Analyses

### TEAEs

The incidence of all TEAEs reported during the study was similar between subjects who received mirikizumab Formulation A-P and mirikizumab Formulation B-P (Table 28). Injection site data was prospectively assessed, with any event relating to an injection site captured as a study endpoint related to ISRs and not recorded as an AE unless that event qualified as an SAE.

TABLE 28
Summary of Adverse Events

| | Number of Subjects with Events | | | |
|---|---|---|---|---|
| | (Percentage of Subjects | | | |
| | with Events) | | | |
| | 200 mg Mirikizumab | | 200 mg Mirikizumab | |
| | Formulation A-P | | Formulation B-P | |
| | (N = 30) | | (N = 30) | |
| All TEAEs | 3 | (10.0%) | 3 | (10.0%) |
| Mild | 2 | (6.7%) | 5 | (16.7%) |
| Moderate | 3 | (10.0%) | 2 | (6.7%) |
| Severe | 0 | | 0 | |
| Treatment-related AEs | 2 | (6.7%) | 1 | (3.3%) |
| Fatal AEs | 0 | | 0 | |
| SAEs | 0 | | 0 | |
| AEs leading to discon- | 0 | | 0 | |
| tinuation from study | | | | |
| Infections | 0 | | 1 | (3.3%) |
| Systemic Allergic/ | 0 | | 0 | |
| Hypersensitivity | | | | |
| Reactions | | | | |
| ISRs | 23 | (76.7%) | 15 | (50.0%) |

Overall, 3 (10.0%) subjects who received mirikizumab Formulation A-P reported a total of 5 TEAEs and 3 (10.0%) subjects who received mirikizumab Formulation B-P reported a total of 7 TEAEs (Tables 29a and 29b). TEAEs that were considered related to mirikizumab were reported as follows:
a) Mirikizumab Formulation A-P (4 events in 2 [6.7%] subjects)
   1 subject had single events of mild nausea, moderate vomiting, and moderate headache
   1 subject had a single event of mild nausea
b) Mirikizumab Formulation B-P (2 events in 1 subject [3.3%])
   1 subject had single events of mild nausea and mild headache

All but one TEAE of a moderate broken heel bone, considered related to other medical condition, had resolved by the end of the study, and the majority resolved without treatment. Two treatment-related TEAEs of headache required paracetamol, and the broken heel bone, required apixaban, hydrocodone, and paracetamol.

TABLE 29a
Summary of Treatment-Emergent Adverse Events for Formulation A-P

| | All causalities | | Related to study treatment | |
|---|---|---|---|---|
| | Number of | | Number of | |
| | subjects [%] | | subjects [%] | |
| | with | Number of | with | Number of |
| | treatment- | treatment- | treatment- | treatment- |
| | emergent | emergent | emergent | emergent |
| | adverse | adverse events | adverse | adverse events |
| Treatment | events | and severity* | events | and severity* |
| Formulation A-P | 0 [0.0%] | Mild (0) | 0 [0.0%] | Mild (0) |
| (Arm) | | Moderate (0) | | Moderate (0) |
| (N = 10) | | Severe (0) | | Severe (0) |
| | | Total (0) | | Total (0) |
| Formulation A-P | 2 [20.0%] | Mild (2) | 2 [20.0%] | Mild (2) |
| (Thigh) | | Moderate (2) | | Moderate (2) |
| (N = 10) | | Severe (0) | | Severe (0) |
| | | Total (4) | | Total (4) |
| Formulation A-P | 1 [10.0%] | Mild (0) | 0 [0.0%] | Mild (0) |
| (Abdomen) | | Moderate (1) | | Moderate (0) |
| (N = 10) | | Severe (0) | | Severe (0) |
| | | Total (1) | | Total (0) |

*Only the maximum severity of each adverse event is reported

TABLE 29b
Summary of Treatment-Emergent Adverse Events for Formulation B-P

| | All causalities | | Related to study treatment | |
|---|---|---|---|---|
| | Number of | | Number of | |
| | subjects [%] | | subjects [%] | |
| | with | Number of | with | Number of |
| | treatment- | treatment- | treatment- | treatment- |
| | emergent | emergent | emergent | emergent |
| | adverse | adverse events | adverse | adverse events |
| Treatment | events | and severity* | events | and severity* |
| Formulation B-P | 0 [0.0%] | Mild (0) | 0 [0.0%] | Mild (0) |
| (Arm) | | Moderate (0) | | Moderate (0) |
| (N = 10) | | Severe (0) | | Severe (0) |
| | | Total (0) | | Total (0) |
| Formulation B-P | 0 [0.0%] | Mild (0) | 0 [0.0%] | Mild (0) |
| (Thigh) | | Moderate (0) | | Moderate (0) |
| (N = 10) | | Severe (0) | | Severe (0) |
| | | Total (0) | | Total (0) |
| Formulation B-P | 3 [30.0%] | Mild (5) | 1 [10.0%] | Mild (2) |
| (Abdomen) | | Moderate (2) | | Moderate (0) |
| (N = 10) | | Severe (0) | | Severe (0) |
| | | Total (7) | | Total (2) |

*Only the maximum severity of each adverse event is reported

### Deaths, SAEs and Discontinuations

No deaths occurred during the study. No SAEs occurred during the study. There were no discontinuations due to AEs during the study.

### Injection Site Assessments

Injection-site bleeding was reported in 3 (10.0%) subjects who received mirikizumab Formulation A-P (2 arm, 1 abdomen) and 3 (10.0%) subjects who received mirikizumab Formulation B-P (2 arm, 1 thigh).

The first injection site for each subject was assessed prospectively for ISRs at the time points indicated above. The injection site was assessed for erythema, edema, induration, pruritus, and pain, with each positive response in any category at each time point counted as an event. In addition, any spontaneously reported ISR at either the first or second injection site was assessed as above.

Injection site reaction data are summarized in Tables 30a and 30b. This includes data from the planned prospective assessments and assessment of ISRs spontaneously reported at each injection site on Day 9 by 1 subject who received Formulation A-P (arm).

TABLE 30a
Summary of Injection Site Reaction Data for Formulation A-P

| | | Formulation | | Formulation | | Formulation | |
|---|---|---|---|---|---|---|---|
| | | A-P | | A-P | | A-P | |
| | | (Arm) | | (Thigh) | | (Abdomen) | |
| Parameter | | (N = 10) | | (N = 10) | | (N = 10) | |
| Number (%) of | | 7 | (70.0%) | 8 | (80.0%) | 8 | (80.0%) |
| subjects reporting | | | | | | | |
| ISRs | | | | | | | |
| Number of | | 19 | | 14 | | 14 | |
| reported ISRs | | | | | | | |
| Time of ISR | During administration | 0 | (0.0%) | 0 | (0.0%) | 0 | (0.0%) |
| relative to study | Within 30 minutes of | 13 | (68.4%) | 12 | (85.7%) | 13 | (92.9%) |
| drug | administration | | | | | | |
| administration* | >30 minutes and up to 6 | 0 | (0.0%) | 2 | (14.3%) | 1 | (7.1%) |
| | hours after | | | | | | |
| | administration | | | | | | |
| | >6 hours and up to 24 | 0 | (0.0%) | 0 | (0.0%) | 0 | (0.0%) |
| | hours after | | | | | | |
| | administration | | | | | | |
| | >24 hours and up to 14 | 6 | (31.6%) | 0 | (0.0%) | 0 | (0.0%) |
| | days after administration | | | | | | |
| | >14 days after | 0 | (0.0%) | 0 | (0.0%) | 0 | (0.0%) |
| | administration | | | | | | |
| | Unknown | 0 | (0.0%) | 0 | (0.0%) | 0 | (0.0%) |
| Size of | Barely Noticeable (less | 6 | (31.6%) | 5 | (35.7%) | 4 | (28.6%) |
| erythema* | than 25 mm diameter) | | | | | | |
| | Slight (25-50 mm | 2 | (10.5%) | 0 | (0.0%) | 0 | (0.0%) |
| | diameter) | | | | | | |
| | Moderate (51-100 mm | 1 | (5.3%) | 0 | (0.0%) | 0 | (0.0%) |
| | diameter) | | | | | | |
| | Severe (more than 100 | 0 | (0.0%) | 0 | (0.0%) | 0 | (0.0%) |
| | mm diameter) | | | | | | |
| Severity of | Noticeable but very mild redness | 6 | (31.6%) | 5 | (35.7%) | 4 | (28.6%) |
| erythema* | Clearly red | 3 | (15.8%) | 0 | (0.0%) | 0 | (0.0%) |
| | Bright red | 0 | (0.0%) | 0 | (0.0%) | 0 | (0.0%) |
| | Dark with ulceration, or | 0 | (0.0%) | 0 | (0.0%) | 0 | (0.0%) |
| | necrosis | | | | | | |
| Severity of | Barely Noticeable (less than | 2 | (10.5%) | 0 | (0.0%) | 0 | (0.0%) |
| induration* | 25 mm diameter) | | | | | | |
| | Slight (25-50 mm | 0 | (0.0%) | 0 | (0.0%) | 0 | (0.0%) |
| | diameter) | | | | | | |
| | Moderate (51-100 | 0 | (0.0%) | 0 | (0.0%) | 0 | (0.0%) |
| | mm diameter) | | | | | | |
| | Severe (more than | 0 | (0.0%) | 0 | (0.0%) | 0 | (0.0%) |
| | 100 mm diameter) | | | | | | |
| Did the subject | Yes | 6 | (31.6%) | 9 | (64.3%) | 10 | (71.4%) |
| have injection site | No | 13 | (68.4%) | 5 | (35.7%) | 4 | (28.6%) |
| pain?* | | | | | | | |
| Severity of | Mild | 2 | (10.5%) | 0 | (0.0%) | 0 | (0.0%) |
| pruritus* | Moderate | 0 | (0.0%) | 0 | (0.0%) | 0 | (0.0%) |
| | Severe | 0 | (0.0%) | 0 | (0.0%) | 0 | (0.0%) |
| Severity of | Mild (less than 2 mm) | 0 | (0.0%) | 0 | (0.0%) | 0 | (0.0%) |
| edema* | Moderate (2-5 mm) | 0 | (0.0%) | 0 | (0.0%) | 0 | (0.0%) |
| | Severe (more than 5 mm) | 0 | (0.0%) | 0 | (0.0%) | 0 | (0.0%) |

*Percentages are based on number of reported ISRs
Subjects with a change in severity in ISRs are only counted one time at the highest severity

TABLE 30b
Summary of Injection Site Reaction Data for Formulation B-P

| | | Formulation | | Formulation | | Formulation | |
|---|---|---|---|---|---|---|---|
| | | B-P | | B-P | | B-P | |
| | | (Arm) | | (Thigh) | | (Abdomen) | |
| Parameter | | (N = 10) | | (N = 10) | | (N = 10) | |
| Number (%) of | | 6 | (60.0%) | 4 | (40.0%) | 5 | (50.0%) |
| subjects with | | | | | | | |
| ISRs | | | | | | | |
| Number of ISRs | | 7 | | 6 | | 7 | |
| Time of ISR | During administration | 0 | (0.0%) | 0 | (0.0%) | 0 | (0.0%) |
| relative to study | Within 30 minutes of | 5 | (71.4%) | 5 | (83.3%) | 7 | (100.0%) |
| drug | administration | | | | | | |
| administration* | >30 minutes and up to | 2 | (28.6%) | 1 | (16.7%) | 0 | (0.0%) |
| | 6 hours after | | | | | | |
| | administration | | | | | | |
| | >6 hours and up to | 0 | (0.0%) | 0 | (0.0%) | 0 | (0.0%) |
| | 24 hours after | | | | | | |
| | administration | | | | | | |
| | >24 hours and up to | 0 | (0.0%) | 0 | (0.0%) | 0 | (0.0%) |
| | 14 days after | | | | | | |
| | administration | | | | | | |
| | >14 days after | 0 | (0.0%) | 0 | (0.0%) | 0 | (0.0%) |
| | administration | | | | | | |
| Size of | Barely Noticeable | 3 | (42.9%) | 3 | (50.0%) | 1 | (14.3%) |
| erythema* | (less than 25 mm | | | | | | |
| | diameter) | | | | | | |
| | Slight (25-50 mm | 0 | (0.0%) | 0 | (0.0%) | 0 | (0.0%) |
| | diameter) | | | | | | |
| | Moderate (51-100 | 0 | (0.0%) | 0 | (0.0%) | 0 | (0.0%) |
| | mm diameter) | | | | | | |
| | Severe (more than | 0 | (0.0%) | 0 | (0.0%) | 0 | (0.0%) |
| | 100 mm diameter) | | | | | | |
| Severity of | Noticeable but very mild redness | 3 | (42.9%) | 3 | (50.0%) | 1 | (14.3%) |
| erythema* | Clearly red | 0 | (0.0%) | 0 | (0.0%) | 0 | (0.0%) |
| | Bright red | 0 | (0.0%) | 0 | (0.0%) | 0 | (0.0%) |
| | Dark with ulceration | 0 | (0.0%) | 0 | (0.0%) | 0 | (0.0%) |
| Severity of | Barely Noticeable | 0 | (0.0%) | 0 | (0.0%) | 0 | (0.0%) |
| induration* | (less than 25 mm diameter) | | | | | | |
| | Slight (25-50 mm diameter) | 0 | (0.0%) | 0 | (0.0%) | 0 | (0.0%) |
| | Moderate (51-100 mm diameter) | 0 | (0.0%) | 0 | (0.0%) | 0 | (0.0%) |
| | Severe (more than | 0 | (0.0%) | 0 | (0.0%) | 0 | (0.0%) |
| | 100 mm diameter) | | | | | | |
| Did the subject | Yes | 4 | (57.1%) | 3 | (50.0%) | 6 | (85.7%) |
| have injection | No | 3 | (42.9%) | 3 | (50.0%) | 1 | (14.3%) |
| site pain?* | | | | | | | |
| Severity of | Mild | 0 | (0.0%) | 0 | (0.0%) | 0 | (0.0%) |
| pruritus* | Moderate | 0 | (0.0%) | 0 | (0.0%) | 0 | (0.0%) |
| | Severe | 0 | (0.0%) | 0 | (0.0%) | 0 | (0.0%) |
| Severity of | Mild (less than 2 mm) | 0 | (0.0%) | 0 | (0.0%) | 0 | (0.0%) |
| edema* | Moderate (2-5 mm) | 0 | (0.0%) | 0 | (0.0%) | 0 | (0.0%) |
| | Severe (more than 5 mm) | 0 | (0.0%) | 0 | (0.0%) | 0 | (0.0%) |

*Percentages are based on number of reported ISRs
Subjects with a change in severity in ISRs are only counted one time at the highest severity

Overall, 23 (76.7%) subjects who received the mirikizumab Formulation A-P (7 arm, 8 thigh, 8 abdomen) reported 47 ISRs, and 15 (50.0%) subjects who received the mirikizumab Formulation B-P (6 arm, 6 thigh, 6 abdomen) reported 20 ISRs. The number of ISRs were similar between injection sites (arm, thigh, abdomen) for subjects who received the mirikizumab Formulation A-P or mirikizumab Formulation B-P. The majority of reports of ISRs consisted of mild reaction. Most responses (82.1%) were made within 30 minutes of treatment administration.

### Categorical Pain

During assessment of ISRs, subjects were asked whether there was injection site pain (“yes/no”). Following administration of mirikizumab Formulation A-P, 25 events of pain were reported by 22 (73.3%) subjects (6 arm, 8 thigh, 8 abdomen). Following administration of mirikizumab Formulation B-P, 13 events of pain were reported by 11 (36.7%) subjects (4 arm, 3 thigh, 4 abdomen).

### Pain Visual Analog Scale

Reports of injection-site pain were further assessed using the VAS pain assessment. A summary of VAS pain score data by injection site is shown in Tables 31a and 31b.

TABLE 31a
Summary of the VAS Pain Score Data for Formulation A-P

| | | Pain at this time* (mm) | | | |
|---|---|---|---|---|---|
| | | 1 | 5 | 15 | 30 |
| Treatment | | minute | minutes | minutes | minutes |
| Formulation A-P | Mean | 19.7 | 5.0 | 0.8 | NC |
| (Arm) | SD | 17.9 | 7.0 | 1.3 | NC |
| (N = 10) | Median | 16.0 | 1.5 | 0.0 | NC |
| | Minimum | 2 | 0 | 0 | 0 |
| | Maximum | 59 | 18 | 4 | 0 |
| | n | 10 | 10 | 10 | 1 |
| Formulation A-P | Mean | 36.9 | 7.9 | 3.5 | NC |
| (Thigh) | SD | 25.6 | 16.0 | 6.8 | NC |
| (N = 10) | Median | 28.0 | 0.5 | 1.0 | NC |
| | Minimum | 6 | 0 | 0 | 0 |
| | Maximum | 84 | 51 | 22 | 1 |
| | n | 10 | 10 | 10 | 2 |
| Formulation A-P | Mean | 21.6 | 5.1 | 0.6 | NC |
| (Abdomen) | SD | 19.1 | 11.3 | 1.0 | NC |
| (N = 10) | Median | 17.0 | 1.5 | 0.0 | NC |
| | Minimum | 2 | 0 | 0 | 0 |
| | Maximum | 56 | 37 | 2 | 0 |
| | n | 10 | 10 | 10 | 1 |
| Formulation A-P | Mean | 26.1 | 6.0 | 1.6 | 0.3 |
| (Overall)(N = 30) | SD | 21.8 | 11.7 | 4.1 | 0.5 |
| | Median | 22.5 | 1.0 | 0.0 | 0.0 |
| | Minimum | 2 | 0 | 0 | 0 |
| | Maximum | 84 | 51 | 22 | 1 |
| | n | 30 | 30 | 30 | 4 |

NC = Not calculated
*0 mm = No pain and 100 mm = Worst imaginable pain
At time points 30, 60, 120, and 240 minutes, VAS pain assessment occured only if pain was reported as “yes” on the Injection-site Assessment form.

TABLE 31b
Summary of the VAS Pain Score Data for Formulation B-P

| | | Pain at this time* (mm) | | | |
|---|---|---|---|---|---|
| | | 1 | 5 | 15 | 30 |
| Treatment | | minute | minutes | minutes | minutes |
| Formulation B-P | Mean | 14.0 | 1.4 | 0.5 | NC |
| (Arm) | SD | 14.0 | 2.3 | 1.0 | NC |
| (N = 10) | Median | 10.5 | 0.0 | 0.0 | NC |
| | Minimum | 0 | 0 | 0 | 0 |
| | Maximum | 44 | 6 | 3 | 0 |
| | n | 10 | 10 | 10 | 1 |
| Formulation B-P | Mean | 13.1 | 1.3 | 0.9 | |
| (Thigh) | SD | 10.1 | 1.9 | 1.6 | |
| (N = 10) | Median | 11.0 | 1.0 | 0.0 | |
| | Minimum | 3 | 0 | 0 | |
| | Maximum | 36 | 6 | 5 | |
| | n | 10 | 10 | 10 | |
| Formulation B-P | Mean | 10.8 | 3.1 | 1.7 | NC |
| (Abdomen) | SD | 7.1 | 2.7 | 2.0 | NC |
| (N = 10) | Median | 9.5 | 2.5 | 1.0 | NC |
| | Minimum | 0 | 0 | 0 | 0 |
| | Maximum | 26 | 9 | 5 | 0 |
| | n | 10 | 10 | 10 | 1 |
| Formulation B-P | Mean | 12.6 | 1.9 | 1.0 | NC |
| (Overall)(N = 30) | SD | 10.5 | 2.4 | 1.6 | NC |
| | Median | 10.0 | 1.0 | 0.0 | NC |
| | Minimum | 0 | 0 | 0 | 0 |
| | Maximum | 44 | 9 | 5 | 0 |
| | n | 30 | 30 | 30 | 2 |

NC = Not calculated
*0 mm = No pain and 100 mm = Worst imaginable pain
At time points 30, 60, 120, and 240 minutes, VAS pain assessment occured only if pain was reported as “yes” on the Injection-site Assessment form.

Within 1 minute post-dose, mean VAS pain score was 26.1 following administration of mirikizumab Formulation A-P, and 12.6 following administration of mirikizumab Formulation B-P. This difference is statistically significant, with the 90% CIs of the difference in geometric LS means excluding unity (Table 32).

TABLE 32
Statistical Analysis of 1-Minute Pain Measurement using VAS Data

| | | | Difference of | |
|---|---|---|---|---|
| | | Least | least squares mean | 90% CI for |
| | | squares | (Formulation B-P − | the difference |
| Treatment | n | mean | Formulation A-P) | (Lower, Upper) |
| Formulation A-P (Overall) | 30 | 26.07 | | |
| Formulation B-P (Overall) | 30 | 12.63 | −13.43 | (−20.75, −6.12) |
| Formulation A-P (Arm) | 10 | 19.70 | | |
| Formulation B-P (Arm) | 10 | 14.00 | −5.70 | (−18.16, 6.76) |
| Formulation A-P (Thigh) | 10 | 36.90 | | |
| Formulation B-P (Thigh) | 10 | 13.10 | −23.80 | (−38.90, −8.70) |
| Formulation A-P (Abdomen) | 10 | 21.60 | | |
| Formulation B-P (Abdomen) | 10 | 10.80 | −10.80 | (−21.95, 0.35) |

Abbreviations:
CI = confidence interval;
n = number of observations
The VAS scores range from 0 mm (no pain) to 100 mm (worst imaginable pain)

At 5 minute post-dose, mean VAS pain score was 6.0 following administration of mirikizumab Formulation A-P, and 1.9 following administration of mirikizumab Formulation B-P.

Similar findings were observed when the thigh injection site was considered separately, although there was no statistically significant difference in mean VAS pain score between the mirikizumab Formulation A-P and mirikizumab Formulation B-P at the arm and abdomen injection sites. The majority of pain reported was mild in severity.

Severe pain was only reported by 2 subjects who received the mirikizumab Formulation A-P (thigh).

Listing of Sequences
Heavy Chain CDRs
SEQ ID NO: 1
GYKFTRYVMH
SEQ ID NO: 2
YINPYNDGTNYNEKFKG
SEQ ID NO: 3
ARNWDTGL
Light Chain CDRs
SEQ ID NO: 4
KASDHILKFLT
SEQ ID NO: 5
GATSLET
SEQ ID NO: 6
QMYWSTPFT
Heavy Chain Variable Regions
SEQ ID NO: 7
QVQLVQSGAEVKKPGSSVKVSCKASGYKFTRYVMHWVRQAPGQGLEWMGYI
NPYNDGTNYNEKFKGRVTITADKSTSTAYMELSSLRSEDTAVYYCARNWDT
GLWGQGTTVTVSS
Light Chain Variable Regions
SEQ ID NO: 8
DIQMTQSPSSLSASVGDRVTITCKASDHILKFLTWYQQKPGKAPKLLIYGA
TSLETGVPSRFSGSGSGTDFTLTISSLQPEDFATYYCQMYWSTPFTFGGGT
KVEIK
Complete Heavy Chain
SEQ ID NO: 9
QVQLVQSGAEVKKPGSSVKVSCKASGYKFTRYVMHWVRQAPGQGLEWMGYI
NPYNDGTNYNEKFKGRVTITADKSTSTAYMELSSLRSEDTAVYYCARNWDT
GLWGQGTTVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKP
SNTKVDKRVESKYGPPCPPCPAPEAAGGPSVFLFPPKPKDTLMISRTPEVT
CVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVD
KSRWQEGNVFSCSVMHEALHNHYTQKSLSLSLG
Complete Light Chain
SEQ ID NO: 10
DIQMTQSPSSLSASVGDRVTITCKASDHILKFLTWYQQKPGKAPKLLIYGA
TSLETGVPSRFSGSGSGTDFTLTISSLQPEDFATYYCQMYWSTPFTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

1. A pharmaceutical formulation comprising:
(i) 50 mg/mL-150 mg/mL of an IL-23p19 antibody;
(ii) 8 mM-12 mM of a citrate buffer;
(iii) 100-200 mM of sodium chloride (NaCl); and
(iv) 0.01% w/v to 0.05% w/v of a surfactant,
wherein the pH of the formulation is about 5.5, and
wherein the anti-IL-23p19 antibody comprises a light chain variable region (LCVR) and a heavy chain variable region (HCVR), the amino acid sequence of the LCVR is SEQ ID NO: 8 and the amino acid sequence of the HCVR is SEQ ID NO: 7.

2. A pharmaceutical formulation according to claim 1, wherein the anti-IL-23p19 antibody comprises a light chain (LC) and a heavy chain (HC), wherein the amino acid sequence of the LC is SEQ ID NO: 10 and the amino acid sequence of the HC is SEQ ID NO: 9.

3. A pharmaceutical formulation according to claim 1, wherein the anti-IL-23p19 antibody is mirikizumab.

4. A pharmaceutical formulation according to claim 1, wherein the concentration of the anti-IL-23p19 antibody is about 75 mg/mL to about 150 mg/mL.

5. A pharmaceutical formulation according to claim 1, wherein the concentration of the anti-IL-23p19 antibody is about 100 mg/mL to about 150 mg/mL.

6. A pharmaceutical formulation according to claim 1, wherein the concentration of the anti-IL-23p19 antibody is about 100 mg/mL.

7. A pharmaceutical formulation according to claim 1, wherein the concentration of the anti-IL-23p19 antibody is about 125 mg/mL.

8. A pharmaceutical composition according to claim 1, wherein the concentration of the citrate buffer is about 10 mM.

9. A pharmaceutical formulation according to claim 1, wherein the citrate buffer is a sodium citrate buffer.

10. A pharmaceutical formulation according to claim 1, wherein the surfactant is polysorbate 20 or polysorbate 80.

11. A pharmaceutical formulation according to claim 10, wherein the surfactant is polysorbate 80.

12. A pharmaceutical formulation according to claim 1, wherein the concentration of the surfactant is about 0.03% (w/v).

13. A pharmaceutical formulation according to claim 1, wherein the concentration of NaCl is about 150 mM.

14. (canceled)

15. A pharmaceutical formulation according to claim 3, wherein the formulation comprises:
(i) 100 mg/mL or 125 mg/mL of mirikizumab;
(ii) 10 mM of sodium citrate buffer;
(iii) 150 mM of NaCl; and
(iv) 0.03% w/v of polysorbate 80,
wherein the pH of the formulation is about 5.5.

16. A pharmaceutical formulation according to claim 15, wherein the formulation comprises 100 mg/mL of mirikizumab.

17. A pharmaceutical formulation according to claim 15, wherein the formulation comprises 125 mg/mL of mirikizumab.

18. A pharmaceutical formulation comprising:
(i) 50 mg/mL-150 mg/mL of an antibody IL-23p19 antibody;
(ii) 3 mM-12 mM of a histidine buffer;
(iii) 25-75 mM of NaCl;
(iv) 2-5% w/v of a tonicity agent; and
(iv) 0.01% w/v to 0.05% w/v of a surfactant,
wherein the pH of the formulation is between 5.0 to 6.0, and
wherein the anti-IL-23p19 antibody comprises a light chain variable region (LCVR) and a heavy chain variable region (HCVR), the amino acid sequence of the LCVR is SEQ ID NO: 8 and the amino acid sequence of the HCVR is SEQ ID NO: 7.

19. A pharmaceutical formulation according to claim 18, wherein the anti-IL-23p19 antibody comprises a light chain (LC) and a heavy chain (HC), wherein the amino acid sequence of the LC is SEQ ID NO: 10 and the amino acid sequence of the heavy chain is SEQ ID NO: 9.

20. A pharmaceutical formulation according to claim 18, wherein the anti-IL-23p19 antibody is mirikizumab.

21. A pharmaceutical formulation according to claim 18, wherein the concentration of the anti-IL-23p19 antibody is about 75 mg/mL to about 150 mg/mL.

22. A pharmaceutical formulation according to claim 18, wherein the concentration of the anti-IL-23p19 antibody is about 100 mg/mL to about 150 mg/mL.

23. A pharmaceutical formulation according to claim 18, wherein the concentration of the anti-IL-23p19 antibody is about 100 mg/mL.

24. A pharmaceutical formulation according to claim 18, wherein the concentration of the anti-IL-23p19 antibody is about 125 mg/mL.

25. A pharmaceutical composition according to claim 18, wherein the concentration of the histidine buffer is about 5 mM.

26. A pharmaceutical composition according to claim 18, wherein the tonicity agent is mannitol.

27. A pharmaceutical composition according to claim 26, wherein the concentration of mannitol is 3.3% w/v.

28. A pharmaceutical formulation according to claim 18, wherein the surfactant is polysorbate 20 or polysorbate 80.

29. A pharmaceutical formulation according to claim 28, wherein the surfactant is polysorbate 80.

30. A pharmaceutical formulation according to claim 18, wherein the concentration of the surfactant is about 0.03% (w/v).

31. A pharmaceutical formulation according to claim 18, wherein the concentration of NaCl is about 50 mM.

32. A pharmaceutical formulation according to claim 18, wherein the pH of the formulation is about 5.5.

33. A pharmaceutical formulation according to claim 20 comprising:
(i) 100 mg/mL or 125 mg/mL of mirikizumab;
(ii) 5 mM of a histidine buffer;
(iii) 50 mM of NaCl;
(iv) 3.3% w/v of mannitol; and
(v) 0.03% w/v of polysorbate 80,
wherein the pH of the formulation is 5.5.

34. A pharmaceutical formulation according to claim 33, wherein the formulation comprises 100 mg/mL of mirikizumab.

35. A pharmaceutical formulation according to claim 33, wherein the formulation comprises 125 mg/mL of mirikizumab.

36. A method of treating and/or preventing psoriasis, ulcerative colitis, Crohn's Disease, psoriatic arthritis and/or ankylosing spondylitis, wherein the method comprises administering to a patient a therapeutically effective amount of a pharmaceutical formulation of claim 1.

37. A pharmaceutical formulation according to claim 1, for use in the treatment and/or prevention of psoriasis, ulcerative colitis, Crohn's Disease, psoriatic arthritis and/or ankylosing spondylitis.

38. Use of a pharmaceutical formulation according to claim 1, in the manufacture of a medicament for use in the treatment of psoriasis, ulcerative colitis, Crohn's Disease, psoriatic arthritis and/or ankylosing spondylitis.

39. A method of reducing injection-associated pain experienced by a patient at the time of, or shortly after, SC, IP and/or IM administration of a pharmaceutical formulation comprising an anti-IL-23p19 antibody, the method comprising administering to a patient a pharmaceutical formulation according to claim 18, wherein, said step of administering provides a therapeutically favorable level of injection-associated pain.

40. A method of reducing injection-associated pain according to claim 39, wherein the therapeutically favorable level of injection-associated pain comprises a VAS score of less than 30 mm or less than 20 mm.

41. An improved method for SC administration of an anti-IL-23p19 antibody to a patient in need thereof, wherein the improvement comprises a reduction in injection-associated pain upon SC administration of a pharmaceutical formulation comprising an anti-IL-23p19 antibody, the method comprising administering a pharmaceutical formulation according to of claim 18, wherein said step of administering provides an improved level of injection-associated pain and/or provides a therapeutically favorable level of injection-associated pain.

42. An improved method for SC administration of an anti-IL-23p19 antibody according to claim 41, wherein the therapeutically favorable level of injection-associated pain comprises a VAS score of less than 30 mm or less than 20 mm.

43. An improved method of treating at least one of psoriasis, ulcerative colitis, Crohn's Disease, psoriatic arthritis and ankylosing spondylitis, wherein the improvement comprises a reduction in injection-associated pain upon the SC administration of a pharmaceutical formulation comprising an anti-IL-23p19 antibody, the method comprising administering a pharmaceutical formulation according to claim 18, wherein said step of administering provides an improved level of injection-associated pain and/or provides a therapeutically favorable level of injection-associated pain.

44. An improved method of treating at least one of psoriasis, ulcerative colitis, Crohn's Disease, psoriatic arthritis and ankylosing spondylitis according to claim 43, wherein the therapeutically favorable level of injection-associated pain comprises a VAS score of less than 30 mm or less than 20 mm.

45. A method of treating and/or preventing psoriasis, ulcerative colitis, Crohn's Disease, psoriatic arthritis and/or ankylosing spondylitis, wherein the method comprises administering to a patient a therapeutically effective amount of a pharmaceutical formulation of claim 18.

46. A pharmaceutical formulation according to claim 18, for use in the treatment and/or prevention of psoriasis, ulcerative colitis, Crohn's Disease, psoriatic arthritis and/or ankylosing spondylitis.

47. Use of a pharmaceutical formulation according to claim 18, in the manufacture of a medicament for use in the treatment of psoriasis, ulcerative colitis, Crohn's Disease, psoriatic arthritis and/or ankylosing spondylitis.
